(19) United States
(12) Patent Application Publication
(10) Pub. No.: US 2015/0031695 A1
(43) Pub. Date: Jan. 29, 2015

(54) SMALL MOLECULE COMPOUNDS FOR TARGETING INFLAMMATORY CONDITIONS

(71) Applicant: Michael West Brooks, (US)

(72) Inventor: Marvin B. Brooks, Palm Springs, CA (US)

(21) Appl. No.: 14/275,419

(22) Filed: May 12, 2014

Related U.S. Application Data
(63) Continuation of application No. 13/865,507, filed on Apr. 18, 2013, now abandoned.
(60) Provisional application No. 61/636,500, filed on Apr. 20, 2012.

(51) Int. Cl. C07D 239/94; G01N 33/50; C07D 491/056

(52) U.S. Cl. CPC C07D 239/94; C07D 491/056; G01N 33/5023; G01N 2500/04; G01N 2500/10; G01N 2333/525
USPC 514/234.5; 514/266.4; 514/267; 435/29

(57) ABSTRACT

Small molecule compounds for the treatment of inflammatory conditions, and pharmaceutical compositions and methods relating thereto. The compositions may be used for the treatment of conditions such as multiple sclerosis, type 2 diabetes, psoriasis, rheumatoid arthritis, Hashimoto's thyroiditis, and Crohn's disease. In some embodiments, the pharmaceutical composition and methods pertain to small molecule compounds previously known for the treatment of another condition, such as non-small-cell lung cancer (NSCLC).

### INCORPORATION BY REFERENCE TO ANY PRIORITY APPLICATIONS

This application is a continuation of U.S. patent application Ser. No. 13/865,507, filed Apr. 18, 2013, which claims the benefit of U.S. Provisional Patent Application Ser. No. 61/636,500, filed on Apr. 20, 2012, which are incorporated by reference herein in their entireties.

### BACKGROUND OF THE INVENTION

1. Field of the Invention

The subject matter provided herein relates to the use of small molecule compounds for the treatment of inflammatory conditions, including compositions and methods relating to the inhibition of TNF-α.

2. Description of the Related Art

Psoriasis vulgaris is an autoimmune disease of the skin in which the immune system is mistakenly activated, triggering an acceleration of the growth cycle of skin cells. Psoriasis is characterized by sharply demarcated chronic erythematous plaques covered by silvery white scales, most commonly appearing on the elbows, knees, scalp, and torso. Moderate psoriasis is defined as 3% to 10% of the body surface affected, while severe psoriasis is defined as more than 10% of the body surface affected. The plaques of psoriasis are the result of epidermal hyperproliferation with abnormal cellular maturation (Schon M P, Boehncke W H. Psoriasis. N Engl J Med 2005, 352:1899-1912). Tumor necrosis factor-alpha (TNF-α) is prevalent in psoriatic lesions.

Multiple sclerosis (MS) appears to be an autoimmune disease of the central nervous system in which the immune system is mistakenly activated, triggering the destruction of myelin sheaths that insulate axons and allow for efficient neural transmission. In MS, an interplay between inflammatory and neurodegenerative processes results in intermittent neurological disturbances followed by potential progressive disability. TNF-α is present in the lesions and cerebrospinal fluid (CSF) of patients with MS. TNF-α causes oligodendrocyte cell death and is a driving factor in demyelination (Robinson W H, Genovese M C, Moreland L W. Demyelinating and neurologic events reported in association with tumor necrosis factor-a antagonism: by what mechanisms could tumor necrosis factor-a antagonists improve rheumatoid arthritis but exacerbate multiple sclerosis? Arthritis Rheum 2001; 44(9):1977-1983). TNF-α production correlates with MS activity, and the level of TNF-α in the CSF correlates with the degree of disability and rate of deterioration in progressive MS patients (Lim S Y, Constantinescu C S: TNF-α: A paradigm of paradox and complexity in multiple sclerosis and its animal models. Open Autoimmunity J 2010, 2:160-170).

Psoriasis and MS involve aberrant (but different) immune responses, which when triggered lead to chronic inflammation and tissue destruction. In psoriasis, the autoimmune target is normal skin. In MS, the autoimmune target is myelin. Although the clinical manifestations of psoriasis and MS bear no resemblance, they are both autoimmune T-cell mediated diseases which share the genetic IL23R-mediated signaling pathway. But despite this common genetic linkage, psoriasis is not more common in patients with MS (Kwok T, Jing Loo W, Guenther L. Psoriasis and multiple sclerosis: is there a link? J Cutan Med Surg 2010; 14(4):151-155).

Metabolic syndrome is a cluster of cardiovascular risk factors accompanied by a greater than five fold increased risk for developing insulin resistant type 2 diabetes. Metabolic syndrome is characterized by increased visceral fat, which is a source of proinflammatory cytokines, including TNF-α (Saijo N. Population differences in the use of EGFR-targeted agents. Clin Adv Hematol Oncol 2008, 6:730-732). Overexpression of TNF-α plays a role in the pathophysiology of insulin resistance (Lynch T J, Kim E5, Eaby B, Garey J, West D P, Lacouture M E. Epidermal growth factor receptor inhibitor-associated cutaneous toxicities: an evolving paradigm in clinical management. The Oncologist 2007; 12(5):610-621).

Psoriasis is often treated with biologic agents that target TNF-α (Brimhall A K, King L N, Licciardone J C, Jacobe H, Menter A. Safety and efficacy of alefacept, efalizumab, etnaercept and infliximab in treating moderate to severe plaque psoriasis: a meta-analysis of randomized controlled trials. Brit J Derm 2008, 159(2):274-285). However, these protein based drugs are not effective in treating multiple sclerosis because they are unable to penetrate the blood brain barrier and have been associated with neurologic symptoms which may result from an enhanced autoimmune response (Robinson W H, Genovese M C, Moreland L W. Demyelinating and neurologic events reported in association with tumor necrosis factor-a antagonism: by what mechanisms could tumor necrosis factor-a antagonists improve rheumatoid arthritis but exacerbate multiple sclerosis? Arthritis Rheum 2001, 44(9):1977-1983). Further, although several medications are available for the treatment of multiple sclerosis, these treatments can demonstrate adverse effects or be poorly tolerated by patients. A need therefore exists for new therapies for these and other inflammatory conditions, and in particular therapies involving small molecules that are suitable for oral administration.

### SUMMARY OF THE INVENTION

In an embodiment, a method of treating type 2 diabetes in an individual is provided, comprising administering to the individual an effective amount of a composition comprising a compound having the structure of Formula I:

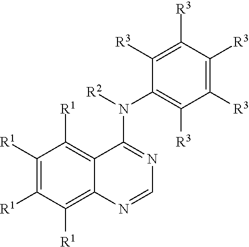

or a pharmaceutically acceptable salt thereof, wherein:
each R¹ is independently hydrogen, hydroxy, halogeno, amino, carboxy, carbamoyl, ureido, (1-4C)alkoxycarbonyl, N-(1-4C)alkylcarbamoyl, N,N-di-[(1-4C)alkyl]carbamoyl, hydroxyamino, (1-4C)alkyl, (1-4C)alkoxy, (1-3C)alkylenedioxy, (1-4C)alkylamino, di-[(1-4C)alkyl]amino, (1-4C)alkylthio, (1-4C)alkyl sulphinyl, (1-4C)alkylsulphonyl, bromomethyl, dibromomethyl, hydroxy-(1-4C)alkyl, (2-4C)alkanoyloxy-(1-4C)alkyl, (1-4C)alkoxy-(1-4C)alkyl, amino-(1-4C)alkyl, (1-4C)alkylamino-(1-4C)alkyl, di-[(1-4C)alkyl]amino-(1-4C)alkyl, piperidino-(1-4C)alkyl, morpholino-(1-4C)alkyl, piperazin-1-yl-(1-4C)alkyl, (1-4C)alkylpiperazin-1-yl-(1-4C)alkyl, hydroxy-(2-4C)alkoxy-(1-4C)alkyl, (1-4C)alkoxy-(2-4C)alkoxy(1-4C)alkyl, hydroxy-(2-4C)alkylamino-(1-4C)alkyl, (1-4C)alkoxy-(2-4C)alkylamino-(1-4C)alkyl, (1-4C)alkylthio-(1-4C)alkyl, hydroxy-(2-4C)alkylthio-(1-4C)alkyl, (1-4C)alkoxy-(2-4C)alkylthio-(1-4C)alkyl, cyano-(1-4C)alkyl, halogeno-(2-4C)alkoxy, hydroxy-(2-4C)alkoxy, (2-4C)alkanoyloxy-(2-4C)alkoxy, (1-4C)alkoxy-(2-4C)alkoxy, carboxy-(1-4C)alkoxy, (1-4C)alkoxycarbonyl-(1-4C)alkoxy, carbamoyl-(1-4C)alkoxy, N-(1-4C)alkylcarbamoyl-(1-4C)alkoxy, N,N-di-[(1-4C)alkyl]carbamoyl-(1-4C)alkoxy, amino-(2-4C)alkoxy, (1-4C)alkylamino-(2-4C)alkoxy, di-[(1-4C)alkyl]amino-(2-4C)alkoxy, piperidino-(2-4C)alkoxy, morpholino-(2-4C)alkoxy, piperazin-1-yl-(2-4C)alkoxy, (1-4C)alkylpiperazin-1-yl-(2-4C)alkoxy, halogeno-(2-4C)alkylamino, hydroxy-(2-4C)alkylamino, (2-4C)alkanoyloxy-(2-4C)alkylamino, (1-4C)alkoxy-(2-4C)alkylamino, amino-(2-4C)alkylamino, (1-4C)alkylamino-(2-4C)alkylamino, di-[(1-4C)alkyl]amino-(2-4C)alkylamino, (2-4C)alkanoylamino, (1-4C)alkyl sulphonylamino, benzamido, benzenesulphonamido, 2-oxopyrrolidin-1-yl, 2,5-dioxopyrrolidin-1-yl, halogeno-(2-4C)alkanoylamino, hydroxy-(2-4C)alkanoylamino, (1-4C)alkoxy-(2-4C)alkanoylamino, carboxy-(2-4C)alkanoylamino, (1-4C)alkoxycarbonyl-(2-4C)alkanoylamino, amino-(2-4C)alkanoylamino, (1-4C)alkylamino-(2-4C)alkanoylamino or di-[(1-4C)alkyl]amino-(2-4C)alkanoylamino, and wherein said benzamido or benzenesulphonamido substituent may optionally bear one or two halogeno, (1-4C)alkyl or (1-4C)alkoxy substituents,
or two R¹ groups are taken together with the carbons to which they are attached to form a 5-15 membered ring that includes 1-6 heteroatoms selected from oxygen, sulfur, and nitrogen;
R² is hydrogen or (1-4C)alkyl;
each R³ is independently hydrogen, hydroxy, halogeno, trifluoromethyl, amino, nitro, cyano, (1-4C)alkyl, (1-4C)alkoxy, H(2-4C)alkynl, or (2-4C)alkenyl;
wherein at least two R¹ are hydrogen; and
wherein at least three R³ are hydrogen.

In an embodiment, method of treating multiple sclerosis in an individual is provided, comprising administering to the individual an effective amount of a composition comprising a compound having the structure of Formula I:

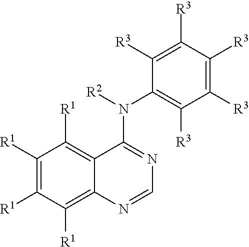

or a pharmaceutically acceptable salt thereof, wherein:
each R¹ is independently hydrogen, hydroxy, halogeno, amino, carboxy, carbamoyl, ureido, (1-4C)alkoxycarbonyl, N-(1-4C)alkylcarbamoyl, N,N-di-[(1-4C)alkyl]carbamoyl, hydroxyamino, (1-4C)alkyl, (1-4C)alkoxy, (1-3C)alkylenedioxy, (1-4C)alkylamino, di-[(1-4C)alkyl]amino, (1-4C)alkylthio, (1-4C)alkyl sulphinyl, (1-4C)alkylsulphonyl, bromomethyl, dibromomethyl, hydroxy-(1-4C)alkyl, (2-4C)alkanoyloxy-(1-4C)alkyl, (1-4C)alkoxy-(1-4C)alkyl, amino-(1-4C)alkyl, (1-4C)alkylamino-(1-4C)alkyl, di-[(1-4C)alkyl]amino-(1-4C)alkyl, piperidino-(1-4C)alkyl, morpholino-(1-4C)alkyl, piperazin-1-yl-(1-4C)alkyl, (1-4C)alkylpiperazin-1-yl-(1-4C)alkyl, hydroxy-(2-4C)alkoxy-(1-4C)alkyl, (1-4C)alkoxy-(2-4C)alkoxy(1-4C)alkyl, hydroxy-(2-4C)alkylamino-(1-4C)alkyl, (1-4C)alkoxy-(2-4C)alkylamino-(1-4C)alkyl, (1-4C)alkylthio-(1-4C)alkyl, hydroxy-(2-4C)alkylthio-(1-4C)alkyl, (1-4C)alkoxy-(2-4C)alkylthio-(1-4C)alkyl, cyano-(1-4C)alkyl, halogeno-(2-4C)alkoxy, hydroxy-(2-4C)alkoxy, (2-4C)alkanoyloxy-(2-4C)alkoxy, (1-4C)alkoxy-(2-4C)alkoxy, carboxy-(1-4C)alkoxy, (1-4C)alkoxycarbonyl-(1-4C)alkoxy, carbamoyl-(1-4C)alkoxy, N-(1-4C)alkylcarbamoyl-(1-4C)alkoxy, N,N-di-[(1-4C)alkyl]carbamoyl-(1-4C)alkoxy, amino-(2-4C)alkoxy, (1-4C)alkylamino-(2-4C)alkoxy, di-[(1-4C)alkyl]amino-(2-4C)alkoxy, piperidino-(2-4C)alkoxy, morpholino-(2-4C)alkoxy, piperazin-1-yl-(2-4C)alkoxy, (1-4C)alkylpiperazin-1-yl-(2-4C)alkoxy, halogeno-(2-4C)alkylamino, hydroxy-(2-4C)alkylamino, (2-4C)alkanoyloxy-(2-4C)alkylamino, (1-4C)alkoxy-(2-4C)alkylamino, amino-(2-4C)alkylamino, (1-4C)alkylamino-(2-4C)alkylamino, di-[(1-4C)alkyl]amino-(2-4C)alkylamino, (2-4C)alkanoylamino, (1-4C)alkyl sulphonylamino, benzamido, benzenesulphonamido, 2-oxopyrrolidin-1-yl, 2,5-dioxopyrrolidin-1-yl, halogeno-(2-4C)alkanoylamino, hydroxy-(2-4C)alkanoylamino, (1-4C)alkoxy-(2-4C)alkanoylamino, carboxy-(2-4C)alkanoylamino, (1-4C)alkoxycarbonyl-(2-4C)alkanoylamino, amino-(2-4C)alkanoylamino, (1-4C)alkylamino-(2-4C)alkanoylamino or di-[(1-4C)alkyl]amino-(2-4C)alkanoylamino, and wherein said benzamido or benzenesulphonamido substituent may optionally bear one or two halogeno, (1-4C)alkyl or (1-4C)alkoxy substituents,
or two R¹ groups are taken together with the carbons to which they are attached to form a 5-15 membered ring that includes 1-6 heteroatoms selected from oxygen, sulfur, and nitrogen;
R² is hydrogen or (1-4C)alkyl;
each R³ is independently hydrogen, hydroxy, halogeno, trifluoromethyl, amino, nitro, cyano, (1-4C)alkyl, (1-4C)alkoxy, H(2-4C)alkynl, or (2-4C)alkenyl;
wherein at least two R¹ are hydrogen; and
wherein at least three R³ are hydrogen.

In an embodiment, method of treating an inflammatory condition in an individual is provided, comprising administering to the individual an effective amount of a composition comprising a compound having the structure of Formula I:

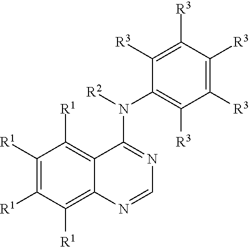

or a pharmaceutically acceptable salt thereof, wherein:
each R¹ is independently hydrogen, hydroxy, halogeno, amino, carboxy, carbamoyl, ureido, (1-4C)alkoxycarbonyl, N-(1-4C)alkylcarbamoyl, N,N-di-[(1-4C)alkyl]carbamoyl, hydroxyamino, (1-4C)alkyl, (1-4C)alkoxy, (1-3C)alkylenedioxy, (1-4C)alkylamino, di-[(1-4C)alkyl]amino, (1-4C)alkylthio, (1-4C)alkyl sulphinyl, (1-4C)alkylsulphonyl, bromomethyl, dibromomethyl, hydroxy-(1-4C)alkyl, (2-4C)alkanoyloxy-(1-4C)alkyl, (1-4C)alkoxy-(1-4C)alkyl, amino-(1-4C)alkyl, (1-4C)alkylamino-(1-4C)alkyl, di-[(1-4C)alkyl]amino-(1-4C)alkyl, piperidino-(1-4C)alkyl, morpholino-(1-4C)alkyl, piperazin-1-yl-(1-4C)alkyl, (1-4C)alkylpiperazin-1-yl-(1-4C)alkyl, hydroxy-(2-4C)alkoxy-(1-4C)alkyl, (1-4C)alkoxy-(2-4C)alkoxy(1-4C)alkyl, hydroxy-(2-4C)alkylamino-(1-4C)alkyl, (1-4C)alkoxy-(2-4C)alkylamino-(1-4C)alkyl, (1-4C)alkylthio-(1-4C)alkyl, hydroxy-(2-4C)alkylthio-(1-4C)alkyl, (1-4C)alkoxy-(2-4C)alkylthio-(1-4C)alkyl, cyano-(1-4C)alkyl, halogeno-(2-4C)alkoxy, hydroxy-(2-4C)alkoxy, (2-4C)alkanoyloxy-(2-4C)alkoxy, (1-4C)alkoxy-(2-4C)alkoxy, carboxy-(1-4C)alkoxy, (1-4C)alkoxycarbonyl-(1-4C)alkoxy, carbamoyl-(1-4C)alkoxy, N-(1-4C)alkylcarbamoyl-(1-4C)alkoxy, N,N-di-[(1-4C)alkyl]carbamoyl-(1-4C)alkoxy, amino-(2-4C)alkoxy, (1-4C)alkylamino-(2-4C)alkoxy, di-[(1-4C)alkyl]amino-(2-4C)alkoxy, piperidino-(2-4C)alkoxy, morpholino-(2-4C)alkoxy, piperazin-1-yl-(2-4C)alkoxy, (1-4C)alkylpiperazin-1-yl-(2-4C)alkoxy, halogeno-(2-4C)alkylamino, hydroxy-(2-4C)alkylamino, (2-4C)alkanoyloxy-(2-4C)alkylamino, (1-4C)alkoxy-(2-4C)alkylamino, amino-(2-4C)alkylamino, (1-4C)alkylamino-(2-4C)alkylamino, di-[(1-4C)alkyl]amino-(2-4C)alkylamino, (2-4C)alkanoylamino, (1-4C)alkyl sulphonylamino, benzamido, benzenesulphonamido, 2-oxopyrrolidin-1-yl, 2,5-dioxopyrrolidin-1-yl, halogeno-(2-4C)alkanoylamino, hydroxy-(2-4C)alkanoylamino, (1-4C)alkoxy-(2-4C)alkanoylamino, carboxy-(2-4C)alkanoylamino, (1-4C)alkoxycarbonyl-(2-4C)alkanoylamino, amino-(2-4C)alkanoylamino, (1-4C)alkylamino-(2-4C)alkanoylamino or di-[(1-4C)alkyl]amino-(2-4C)alkanoylamino, and wherein said benzamido or benzenesulphonamido substituent may optionally bear one or two halogeno, (1-4C)alkyl or (1-4C)alkoxy substituents;
or two R¹ groups are taken together with the carbons to which they are attached to form a 5-15 membered ring that includes 1-6 heteroatoms selected from oxygen, sulfur, and nitrogen;
R² is hydrogen or (1-4C)alkyl;
each R³ is independently hydrogen, hydroxy, halogeno, trifluoromethyl, amino, nitro, cyano, (1-4C)alkyl, (1-4C)alkoxy, H(2-4C)alkynl, or (2-4C)alkenyl;
wherein at least two R¹ are hydrogen;
wherein at least three R³ are hydrogen; and
wherein when Formula I is Formula III, the inflammatory condition is not psoriasis.

In an embodiment, method of evaluating a compound for the treatment of type 2 diabetes is provided, comprising contacting TNF-α with a compound and evaluating the results, wherein the compound has the structure of Formula I:

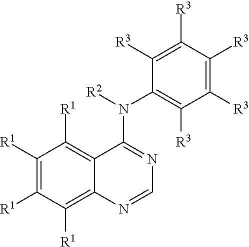

or a pharmaceutically acceptable salt thereof, wherein:
each R¹ is independently hydrogen, hydroxy, halogeno, amino, carboxy, carbamoyl, ureido, (1-4C)alkoxycarbonyl, N-(1-4C)alkylcarbamoyl, N,N-di-[(1-4C)alkyl]carbamoyl, hydroxyamino, (1-4C)alkyl, (1-4C)alkoxy, (1-3C)alkylenedioxy, (1-4C)alkylamino, di-[(1-4C)alkyl]amino, (1-4C)alkylthio, (1-4C)alkyl sulphinyl, (1-4C)alkylsulphonyl, bromomethyl, dibromomethyl, hydroxy-(1-4C)alkyl, (2-4C)alkanoyloxy-(1-4C)alkyl, (1-4C)alkoxy-(1-4C)alkyl, amino-(1-4C)alkyl, (1-4C)alkylamino-(1-4C)alkyl, di-[(1-4C)alkyl]amino-(1-4C)alkyl, piperidino-(1-4C)alkyl, morpholino-(1-4C)alkyl, piperazin-1-yl-(1-4C)alkyl, (1-4C)alkylpiperazin-1-yl-(1-4C)alkyl, hydroxy-(2-4C)alkoxy-(1-4C)alkyl, (1-4C)alkoxy-(2-4C)alkoxy(1-4C)alkyl, hydroxy-(2-4C)alkylamino-(1-4C)alkyl, (1-4C)alkoxy-(2-4C)alkylamino-(1-4C)alkyl, (1-4C)alkylthio-(1-4C)alkyl, hydroxy-(2-4C)alkylthio-(1-4C)alkyl, (1-4C)alkoxy-(2-4C)alkylthio-(1-4C)alkyl, cyano-(1-4C)alkyl, halogeno-(2-4C)alkoxy, hydroxy-(2-4C)alkoxy, (2-4C)alkanoyloxy-(2-4C)alkoxy, (1-4C)alkoxy-(2-4C)alkoxy, carb oxy-(1-4C)alkoxy, (1-4C)alkoxycarbonyl-(1-4C)alkoxy, carbamoyl-(1-4C)alkoxy, N-(1-4C)alkylcarbamoyl-(1-4C)alkoxy, N,N-di-[(1-4C)alkyl]carbamoyl-(1-4C)alkoxy, amino-(2-4C)alkoxy, (1-4C)alkylamino-(2-4C)alkoxy, di-[(1-4C)alkyl]amino-(2-4C)alkoxy, piperidino-(2-4C)alkoxy, morpholino-(2-4C)alkoxy, piperazin-1-yl-(2-4C)alkoxy, (1-4C)alkylpiperazin-1-yl-(2-4C)alkoxy, halogeno-(2-4C)alkylamino, hydroxy-(2-4C)alkylamino, (2-4C)alkanoyloxy-(2-4C)alkylamino, (1-4C)alkoxy-(2-4C)alkylamino, amino-(2-4C)alkylamino, (1-4C)alkylamino-(2-4C)alkylamino, di-[(1-4C)alkyl]amino-(2-4C)alkylamino, (2-4C)alkanoylamino, (1-4C)alkyl sulphonylamino, benzamido, benzenesulphonamido, 2-oxopyrrolidin-1-yl, 2,5-dioxopyrrolidin-1-yl, halogeno-(2-4C)alkanoylamino, hydroxy-(2-4C)alkanoylamino, (1-4C)alkoxy-(2-4C)alkanoylamino, carboxy-(2-4C)alkanoylamino, (1-4C)alkoxycarbonyl-(2-4C)alkanoylamino, amino-(2-4C)alkanoylamino, (1-4C)alkylamino-(2-4C)alkanoylamino or di-[(1-4C)alkyl]amino-(2-4C)alkanoylamino, and wherein said benzamido or benzenesulphonamido substituent may optionally bear one or two halogeno, (1-4C)alkyl or (1-4C)alkoxy substituents;
or two R¹ groups are taken together with the carbons to which they are attached to form a 5-15 membered ring that includes 1-6 heteroatoms selected from oxygen, sulfur, and nitrogen;
R² is hydrogen or (1-4C)alkyl;
each R³ is independently hydrogen, hydroxy, halogeno, trifluoromethyl, amino, nitro, cyano, (1-4C)alkyl, (1-4C)alkoxy, H(2-4C)alkynl, or (2-4C)alkenyl;
wherein at least two R¹ are hydrogen; and
wherein at least three R³ are hydrogen.

In an embodiment, method of evaluating a compound for the treatment of multiple sclerosis is provided, comprising contacting TNF-α with a compound and evaluating the results, wherein the compound has the structure of Formula I:

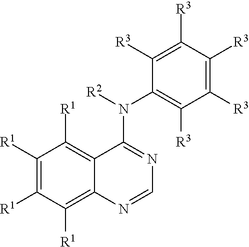

or a pharmaceutically acceptable salt thereof, wherein:
each R¹ is independently hydrogen, hydroxy, halogeno, amino, carboxy, carbamoyl, ureido, (1-4C)alkoxycarbonyl, N-(1-4C)alkylcarbamoyl, N,N-di-[(1-4C)alkyl]carbamoyl, hydroxyamino, (1-4C)alkyl, (1-4C)alkoxy, (1-3C)alkylenedioxy, (1-4C)alkylamino, di-[(1-4C)alkyl]amino, (1-4C)alkylthio, (1-4C)alkyl sulphinyl, (1-4C)alkylsulphonyl, bromomethyl, dibromomethyl, hydroxy-(1-4C)alkyl, (2-4C)alkanoyloxy-(1-4C)alkyl, (1-4C)alkoxy-(1-4C)alkyl, amino-(1-4C)alkyl, (1-4C)alkylamino-(1-4C)alkyl, di-[(1-4C)alkyl]amino-(1-4C)alkyl, piperidino-(1-4C)alkyl, morpholino-(1-4C)alkyl, piperazin-1-yl-(1-4C)alkyl, (1-4C)alkylpiperazin-1-yl-(1-4C)alkyl, hydroxy-(2-4C)alkoxy-(1-4C)alkyl, (1-4C)alkoxy-(2-4C)alkoxy(1-4C)alkyl, hydroxy-(2-4C)alkylamino-(1-4C)alkyl, (1-4C)alkoxy-(2-4C)alkylamino-(1-4C)alkyl, (1-4C)alkylthio-(1-4C)alkyl, hydroxy-(2-4C)alkylthio-(1-4C)alkyl, (1-4C)alkoxy-(2-4C)alkylthio-(1-4C)alkyl, cyano-(1-4C)alkyl, halogeno-(2-4C)alkoxy, hydroxy-(2-4C)alkoxy, (2-4C)alkanoyloxy-(2-4C)alkoxy, (1-4C)alkoxy-(2-4C)alkoxy, carboxy-(1-4C)alkoxy, (1-4C)alkoxycarbonyl-(1-4C)alkoxy, carbamoyl-(1-4C)alkoxy, N-(1-4C)alkylcarbamoyl-(1-4C)alkoxy, N,N-di-[(1-4C)alkyl]carbamoyl-(1-4C)alkoxy, amino-(2-4C)alkoxy, (1-4C)alkylamino-(2-4C)alkoxy, di-[(1-4C)alkyl]amino-(2-4C)alkoxy, piperidino-(2-4C)alkoxy, morpholino-(2-4C)alkoxy, piperazin-1-yl-(2-4C)alkoxy, (1-4C)alkylpiperazin-1-yl-(2-4C)alkoxy, halogeno-(2-4C)alkylamino, hydroxy-(2-4C)alkylamino, (2-4C)alkanoyloxy-(2-4C)alkylamino, (1-4C)alkoxy-(2-4C)alkylamino, amino-(2-4C)alkylamino, (1-4C)alkylamino-(2-4C)alkylamino, di-[(1-4C)alkyl]amino-(2-4C)alkylamino, (2-4C)alkanoylamino, (1-4C)alkyl sulphonylamino, benzamido, benzenesulphonamido, 2-oxopyrrolidin-1-yl, 2,5-dioxopyrrolidin-1-yl, halogeno-(2-4C)alkanoylamino, hydroxy-(2-4C)alkanoylamino, (1-4C)alkoxy-(2-4C)alkanoylamino, carboxy-(2-4C)alkanoylamino, (1-4C)alkoxycarbonyl-(2-4C)alkanoylamino, amino-(2-4C)alkanoylamino, (1-4C)alkylamino-(2-4C)alkanoylamino or di-[(1-4C)alkyl]amino-(2-4C)alkanoylamino, and wherein said benzamido or benzenesulphonamido substituent may optionally bear one or two halogeno, (1-4C)alkyl or (1-4C)alkoxy substituents;
or two R¹ groups are taken together with the carbons to which they are attached to form a 5-15 membered ring that includes 1-6 heteroatoms selected from oxygen, sulfur, and nitrogen;
R² is hydrogen or (1-4C)alkyl;
each R³ is independently hydrogen, hydroxy, halogeno, trifluoromethyl, amino, nitro, cyano, (1-4C)alkyl, (1-4C)alkoxy, H(2-4C)alkynl, or (2-4C)alkenyl;
wherein at least two R¹ are hydrogen; and
wherein at least three R³ are hydrogen.

In an embodiment, a method of evaluating a compound for the treatment of an inflammatory condition is provided, comprising contacting TNF-α with a compound and evaluating the results, wherein the compound has the structure of Formula I:

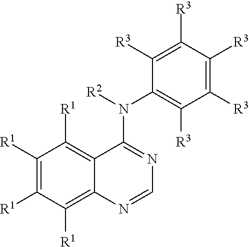

or a pharmaceutically acceptable salt thereof, wherein:
each R¹ is independently hydrogen, hydroxy, halogeno, amino, carboxy, carbamoyl, ureido, (1-4C)alkoxycarbonyl, N-(1-4C)alkylcarbamoyl, N,N-di-[(1-4C)alkyl]carbamoyl, hydroxyamino, (1-4C)alkyl, (1-4C)alkoxy, (1-3C)alkylenedioxy, (1-4C)alkylamino, di-[(1-4C)alkyl]amino, (1-4C)alkylthio, (1-4C)alkyl sulphinyl, (1-4C)alkylsulphonyl, bromomethyl, dibromomethyl, hydroxy-(1-4C)alkyl, (2-4C)alkanoyloxy-(1-4C)alkyl, (1-4C)alkoxy-(1-4C)alkyl, amino-(1-4C)alkyl, (1-4C)alkylamino-(1-4C)alkyl, di-[(1-4C)alkyl]amino-(1-4C)alkyl, piperidino-(1-4C)alkyl, morpholino-(1-4C)alkyl, piperazin-1-yl-(1-4C)alkyl, (1-4C)alkylpiperazin-1-yl-(1-4C)alkyl, hydroxy-(2-4C)alkoxy-(1-4C)alkyl, (1-4C)alkoxy-(2-4C)alkoxy(1-4C)alkyl, hydroxy-(2-4C)alkylamino-(1-4C)alkyl, (1-4C)alkoxy-(2-4C)alkylamino-(1-4C)alkyl, (1-4C)alkylthio-(1-4C)alkyl, hydroxy-(2-4C)alkylthio-(1-4C)alkyl, (1-4C)alkoxy-(2-4C)alkylthio-(1-4C)alkyl, cyano-(1-4C)alkyl, halogeno-(2-4C)alkoxy, hydroxy-(2-4C)alkoxy, (2-4C)alkanoyloxy-(2-4C)alkoxy, (1-4C)alkoxy-(2-4C)alkoxy, carb oxy-(1-4C)alkoxy, (1-4C)alkoxycarbonyl-(1-4C)alkoxy, carbamoyl-(1-4C)alkoxy, N-(1-4C)alkylcarbamoyl-(1-4C)alkoxy, N,N-di-[(1-4C)alkyl]carbamoyl-(1-4C)alkoxy, amino-(2-4C)alkoxy, (1-4C)alkylamino-(2-4C)alkoxy, di-[(1-4C)alkyl]amino-(2-4C)alkoxy, piperidino-(2-4C)alkoxy, morpholino-(2-4C)alkoxy, piperazin-1-yl-(2-4C)alkoxy, (1-4C)alkylpiperazin-1-yl-(2-4C)alkoxy, halogeno-(2-4C)alkylamino, hydroxy-(2-4C)alkylamino, (2-4C)alkanoyloxy-(2-4C)alkylamino, (1-4C)alkoxy-(2-4C)alkylamino, amino-(2-4C)alkylamino, (1-4C)alkylamino-(2-4C)alkylamino, di-[(1-4C)alkyl]amino-(2-4C)alkylamino, (2-4C)alkanoylamino, (1-4C)alkyl sulphonylamino, benzamido, benzenesulphonamido, 2-oxopyrrolidin-1-yl, 2,5-dioxopyrrolidin-1-yl, halogeno-(2-4C)alkanoylamino, hydroxy-(2-4C)alkanoylamino, (1-4C)alkoxy-(2-4C)alkanoylamino, carboxy-(2-4C)alkanoylamino, (1-4C)alkoxycarbonyl-(2-4C)alkanoylamino, amino-(2-4C)alkanoylamino, (1-4C)alkylamino-(2-4C)alkanoylamino or di-[(1-4C)alkyl]amino-(2-4C)alkanoylamino, and wherein said benzamido or benzenesulphonamido substituent may optionally bear one or two halogeno, (1-4C)alkyl or (1-4C)alkoxy substituents,
or two R¹ groups are taken together with the carbons to which they are attached to form a 5-15 membered ring that includes 1-6 heteroatoms selected from oxygen, sulfur, and nitrogen;
R² is hydrogen or (1-4C)alkyl;
each R³ is independently hydrogen, hydroxy, halogeno, trifluoromethyl, amino, nitro, cyano, (1-4C)alkyl, (1-4C)alkoxy, H(2-4C)alkynl, or (2-4C)alkenyl;
wherein at least two R¹ are hydrogen;
wherein at least three R³ are hydrogen; and
wherein when Formula I is Formula III, the inflammatory condition is not psoriasis.

In some embodiments, the method further comprises identifying an individual in need of treatment for type 2 diabetes prior to the administration of the effective amount of a composition comprising a compound having the structure of Formula I.

In some embodiments, the method further comprises identifying an individual in need of treatment for multiple sclerosis prior to the administration of the effective amount of a composition comprising a compound having the structure of Formula I.

In some embodiments, the method further comprises identifying an individual in need of treatment for an inflammatory condition prior to the administration of the effective amount of a composition comprising a compound having the structure of Formula I.

In some embodiments, each R¹ is independently hydrogen, (1-4C)alkoxy, (1-4C)alkoxy-(2-4C)alkoxy, or morpholino-(2-4C)alkoxy;
or two R¹ groups are taken together with the carbons to which they are attached to form a 5-15 membered ring that includes 1-6 heteroatoms selected from oxygen, sulfur, and nitrogen;
R² is hydrogen; and
R³ is hydrogen, halogeno, or H(2-4C)alkynl.

In some embodiments, the inflammatory condition is rheumatoid arthritis.

In some embodiments, the inflammatory condition is Crohn's disease.

In some embodiments, the inflammatory condition is psoriasis.

In some embodiments, the inflammatory condition is moderate psoriasis.

In some embodiments, the inflammatory condition is severe psoriasis.

In some embodiments, the inflammatory condition is Hashimoto's thyroiditis.

In some embodiments, the composition is administered for a period of at least about 4 weeks.

In some embodiments, the composition is administered for a period of at least about 10 weeks.

In some embodiments, the composition is administered for a period of at least about 24 weeks.

In some embodiments, the composition is in oral dosage form.

In some embodiments, the composition is in a sustained-release formulation.

In some embodiments, the daily dosage of the compound is about 50 mg to about 200 mg per day.

In some embodiments, the daily dosage of the compound is about 150 mg per day.

In some embodiments, the compound has the structure of Formula II:

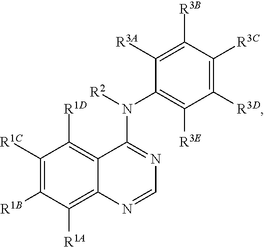

wherein R^{1A} and R^{1D} are hydrogen;
wherein R^{1B} and R^{1C} are methoxyethoxy, methoxy, morpholino-4-yl-propoxy, or wherein R^{1B} and R^{1C} are taken together with the carbons to which they are attached to form a 12 membered ring that includes 4 oxygen heteroatoms;
wherein R^{3A}, R^{3B}, and R^{3E} are hydrogen;
wherein R^{3C} is hydrogen or halogeno; and
wherein R^{3D} is halogeno or ethynyl.

In some embodiments, R^{1B} is methoxy.

In some embodiments, R^{1C} is morpholino-4-yl-propoxy.

In some embodiments, R^{1B} and R^{1C} are methoxyethoxy.

In some embodiments, R^{3C} is hydrogen.

In some embodiments, R^{3C} is halogeno.

In some embodiments, R^{3C} is fluorine.

In some embodiments, R^{3D} is halogeno.

In some embodiments, R^{3D} is chlorine.

In some embodiments, R^{3D} is ethynyl.

In some embodiments, R^{1B} and R^{1C} are taken together with the carbons to which they are attached to form a 12 membered ring that includes 4 oxygen heteroatoms.

In some embodiments, the compound has the structure of Formula III or a pharmaceutically acceptable salt thereof:

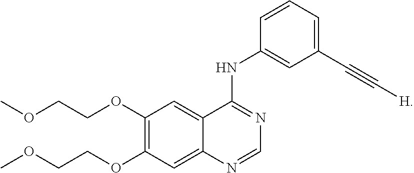

In some embodiments, the compound has the structure of Formula IV or a pharmaceutically acceptable salt thereof:

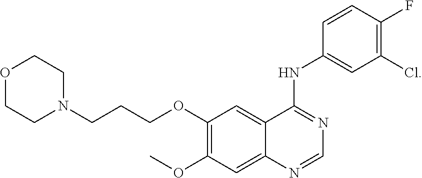 [SMILES: COc1cc2ncnc(Nc3ccc(F)c(Cl)c3)c2cc1OCCCN1CCOCC1]

In some embodiments, the compound has the structure of Formula V or a pharmaceutically acceptable salt thereof:

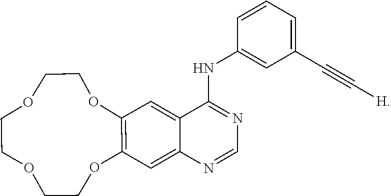

### DETAILED DESCRIPTION OF THE PREFERRED EMBODIMENT

Provided herein are compositions and methods relating to the inhibition of inflammatory conditions. Some of the compounds described herein are cancer therapeutics that are unexpectedly effective in treating inflammatory conditions. For example, as described herein, erlotinib has been found to be unexpectedly effective in treating psoriasis, type 2 diabetes, and Hashimoto's disease.

Erlotinib is an oral agent that is used as a primary therapy of non-small-cell lung cancer (NSCLC) associated with specific epidermal growth factor receptor (EGFR) mutations. Erlotinib is known to act as an EGFR tyrosine kinase inhibitor, and was one of the first orally active, small molecule targeted anticancer drugs approved in the United States (marketed as TARCEVA®). The most common adverse effect of erlotinib is a mild to moderate reversible papulopustular skin eruption on the face, scalp, neck, and back.

Erlotinib's potential role in the treatment of the conditions described herein was previously unknown. For example, previous studies evaluating the effectiveness of erlotinib in NSCLC failed to provide information regarding a possible benefit in MS, as patients with neurologic disorders were excluded (Brimhall A K, King L N, Licciardone J C, Jacobe H, Menter A. Safety and efficacy of alefacept, efalizumab, etnaercept and infliximab in treating moderate to severe plaque psoriasis: a meta-analysis of randomized controlled trials. Brit J Derm 2008, 159(2):274-285).

Without being bound to any particular theory, Applicants believe that the paradox of how erlotinib can produce the remission of NSCLC, the formation of toxic cutaneous papulopustular eruption, and the remission of the inflammatory diseases described herein may be explained by erlotinib having two distinct mechanisms of action—as an EGFR inhibitor and as a TNF-α inhibitor.

In some embodiments, erlotinib controls the conditions described herein by inhibiting TNF-α. In some embodiments, erlotinib alternatively or additionally controls these conditions through its action as an EGFR inhibitor. For example, erlotinib may control hyperproliferative disorders of the epidermis through EGFR inhibition. Erlotinib may also alternatively or additionally control the conditions described herein through a T cell mediated response, RIP2 inhibition, or inhibition of the PI3K/AKT/mTOR pathway. One of skill in the art will recognize that these molecules and pathways, and the conditions related to their dysregulation, are potential targets for the compositions described herein.

Erlotinib is related to other small molecule compounds used in the treatment of NSCLC, including gefitinib (approved and marketed in the United States as IRESSA®) and icotinib (approved and marketed in China as CONMANA®). The compositions and methods described herein comprise these and other structurally similar compounds known to those of skill in the art. In some embodiments, the structurally similar compounds are known to be effective in the treatment of NSCLC or another cancer.

In some embodiments, the compositions described herein are useful for the treatment of a condition associated with the dysregulation of TNF-α. In some embodiments, the condition is selected from the group consisting of type 2 diabetes, MS, rheumatoid arthritis, ankylosing spondylitis, inflammatory bowel disease (such as Crohn's disease), or refractory asthma. For example, in some embodiments, the compositions described herein are used to reduce inflammation and prevent the disease progression of MS.

In some embodiments, the compositions described herein are useful for the treatment of an inflammatory condition associated with the dysregulation of EGFR. For example, in some embodiments, the compositions described herein are used as tyrosine-kinase inhibitors of EGFR.

In some embodiments, the compositions described herein are useful for the treatment of a condition associated with an autoimmune T-cell mediated disease. For example, in some embodiments, the compositions described herein are used in the treatment of psoriasis.

The compositions and methods provided herein include a compound having the structure of Formula I:

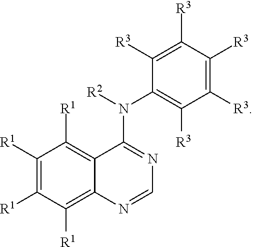

The compositions and methods provided herein include a compound having the structure of Formula II:

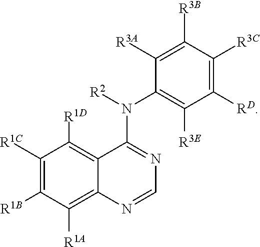

The compositions and methods provided herein include a compound having the structure of Formula III:

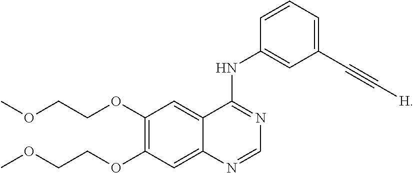

The compositions and methods provided herein include a compound having the structure of Formula IV:

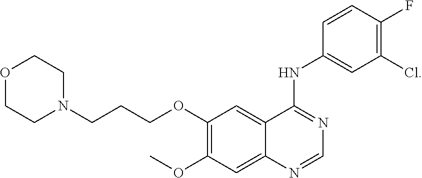 [SMILES: COc1cc2ncnc(Nc3ccc(F)c(Cl)c3)c2cc1OCCCN1CCOCC1]

The compositions and methods provided herein include a compound having the structure of Formula V:

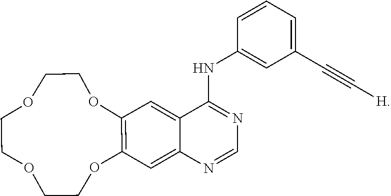

Unless specific definitions are provided, the nomenclatures utilized in connection with, and the laboratory procedures and techniques of, analytical chemistry, synthetic organic chemistry, and medicinal and pharmaceutical chemistry described herein are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, pharmaceutical preparations, formulations, delivery, and the treatment of patients.

In some embodiments, each R¹ is independently hydrogen, hydroxy, amino, carboxy, carbamoyl, ureido, (1-4C)alkoxycarbonyl, N-(1-4C)alkylcarbamoyl, N,N-di-[(1-4C)alkyl]carbamoyl, hydroxyamino, (1-4C)alkoxyamino, (2-4C)alkanoyloxyamino, trifluoromethoxy, (1-4C)alkyl, (1-4C)alkoxy, (1-3C)alkylenedioxy, (1-4C)alkylamino, di-[(1-4C)alkyl]amino, pyrrolidin-1-yl, piperidino, morpholino, piperazin-1-yl, 4-(1-4C)alkylpiperazin-1-yl, (1-4C)alkylthio, (1-4C)alkyl sulphinyl, (1-4C)alkyl sulphonyl, halogeno-(1-4C)alkyl (other than trifluoromethyl), hydroxy-(1-4C)alkyl, (2-4C)alkanoyloxy-(1-4C)alkyl, (1-4C)alkoxy-(1-4C)alkyl, carboxy-(1-4C)alkyl, (1-4C)alkoxycarbonyl-(1-4C)alkyl, carbamoyl-(1-4C)alkyl, N-(1-4C)alkylcarbamoyl-(1-4C)alkyl, N,N-di-[(1-4C)alkyl]-carbamoyl-(1-4C)alkyl, amino-(1-4C)alkyl, (1-4C)alkylamino-(1-4C)alkyl, di-[(1-4C)alkyl]amino-(1-4C)alkyl, piperidino-(1-4C)alkyl, morpholino-(1-4C)alkyl, piperazin-1-yl-(1-4C)alkyl, 4-(1-4C)alkylpiperazin-1-yl-(1-4C)alkyl, hydroxy-(2-4C)alkoxy-(1-4C)alkyl, (1-4C)alkoxy-(2-4C)alkoxy-(1-4C)alkyl, hydroxy-(2-4C)alkylamino-(1-4C)alkyl, (1-4C)alkoxy-(2-4C)alkylamino-(1-4C)alkyl, (1-4C)alkylthio-(1-4C)alkyl, hydroxy-(2-4C)alkylthio-(1-4C)alkyl, (1-4C)alkoxy-(2-4C)alkylthio-(1-4C)alkyl, phenoxy-(1-4C)alkyl, anilino-(1-4C)alkyl, phenylthio-(1-4C)alkyl, cyano-(1-4C)alkyl, halogeno-(2-4C)alkoxy, hydroxy-(2-4C)alkoxy, (2-4C)alkanoyloxy-(2-4C)alkoxy, (1-4C)alkoxy-(2-4C)alkoxy, carboxy-(1-4C)alkoxy, (1-4C)alkoxycarbonyl-(1-4C)alkoxy, carbamoyl-(1-4C)alkoxy, N-(1-4C)alkylcarbamoyl-(1-4C)alkoxy, N,N-di-[(1-4C)alkyl]carbamoyl-(1-4C)alkoxy, amino-(2-4C)alkoxy, (1-4C)alkylamino-(2-4C)alkoxy, di-[(1-4C)alkyl]amino-(2-4C)alkoxy, (2-4C)alkanoyloxy, hydroxy-(2-4C)alkanoyloxy, (1-4C)alkoxy-(2-4C)alkanoyloxy, phenyl-(1-4C)alkoxy, phenoxy-(2-4C)alkoxy, anilino-(2-4C)alkoxy, phenylthio-(2-4C)alkoxy, piperidino-(2-4C)alkoxy, morpholino-(2-4C)alkoxy, piperazin-1-yl-(2-4C)alkoxy, 4-(1-4C)alkylpiperazin-1-yl-(2-4C)alkoxy, halogeno-(2-4C)alkylamino, hydroxy-(2-4C)alkylamino, (2-4C)alkanoyloxy-(2-4C)alkylamino, (1-4C)alkoxy-(2-4C)alkylamino, carboxy-(1-4C)alkylamino, (1-4C)alkoxycarbonyl-(1-4C)alkylamino, carbamoyl-(1-4C)alkylamino, N-(1-4C)alkylcarbamoyl-(1-4C)alkylamino, N,N-di-[(1-4C)alkyl]carbamoyl-(1-4C)alkylamino, amino-(2-4C)alkylamino, (1-4C)alkylamino-(2-4C)alkylamino, di-[(1-4C)alkyl]amino-(2-4C)alkylamino, phenyl-(1-4C)alkylamino, phenoxy-(2-4C)alkylamino, anilino-(2-4C)alkylamino, phenylthio-(2-4C)alkylamino, (2-4C)alkanoylamino, (1-4C)alkoxycarbonylamino, (1-4C)alkylsulphonylamino, benzamido, benzenesulphonamido, 3-phenylureido, 2-oxopyrrolidin-1-yl, 2,5-dioxopyrrolidin-1-yl, halogeno-(2-4C)alkanoylamino, hydroxy-(2-4C)alkanoylamino, (1-4C)alkoxy-(2-4C)alkanoylamino, carboxy-(2-4C)alkanoylamino, (1-4C)alkoxycarbonyl-(2-4C)alkanoylamino, carbamoyl-(2-4C)alkanoylamino, N-(1-4C)alkylcarbamoyl-(2-4C)alkanoylamino, N,N-di-[(1-4C)alkyl]carbamoyl-(2-4C)alkanoylamino, amino-(2-4C)alkanoylamino, (1-4C)alkylamino-(2-4C)alkanoylamino or di-[(1-4C)alkyl]amino-(2-4C)alkanoylamino, and wherein said benzamido or benzenesulphonamido substituent or any anilino, phenoxy or phenyl group in a R¹ substituent may optionally bear one or two halogeno, (1-4C)alkyl or (1-4C)alkoxy substituents.

In some embodiments, each R³ is independently hydrogen, hydroxy, halogeno, trifluoromethyl, amino, nitro, cyano, (1-4C)alkyl, (1-4C)alkoxy, (1-4C)alkylamino, di-[(1-4C)alkyl]amino, (1-4C)alkylthio, (1-4C)alkyl sulphinyl or (1-4C)alkyl sulphonyl; or a pharmaceutically-acceptable salt thereof; except that 4-(4′-hydroxyanilino)-6-methoxyquinazoline, 4-(4′-hydroxyanilino)-6,7-methylenedioxyquinazoline, 4-(4′-hydroxyanilino)-6,7,8-trimethoxyquinazoline, 6-amino-4-(4′-aminoanilino)quinazoline, 4-anilino-6-methylquinazoline or the hydrochloride salt thereof and 4-anilino-6,7-dimethoxyquinazoline or the hydrochloride salt thereof are excluded.

In some embodiments, each R¹ is independently selected from the group consisting of hydrogen, halo, hydroxy, hydroxyamino, carboxy, nitro, guanidino, ureido, cyano, trifluoromethyl, and —(C₁-C₄ alkylene)-W-(phenyl) wherein W is a single bond, O, S or NH.

In some embodiments, each R¹ is independently selected from R⁹ and C₁-C₄ alkyl substituted by cyano, wherein R⁹ is selected from the group consisting of R⁵, —OR⁶, —NR⁶R⁶, —C(O)R⁷, —NHOR⁵, —OC(O)R⁶, cyano, A and —YR⁵; R⁵ is C₁-C₄ alkyl; R⁶ is independently hydrogen or R⁵; R⁷ is R⁵, —OR⁶ or —NR⁶R⁶; A is selected from piperidino, morpholino, pyrrolidino, 4-R⁶-piperazin-1-yl, imidazol-1-yl, 4-pyridon-1-yl, —(C₁-C₄ alkylene)(CO₂H), phenoxy, phenyl, phenylsulfanyl, C₂-C₄ alkenyl, and —(C₁-C₄ alkylene)C(O)NR⁶R⁶; and Y is S, SO, or SO₂; wherein the alkyl moieties in R⁵, —OR⁶ and —NR⁶ R⁶ are optionally substituted by one to three halo substituents and the alkyl moieties in R⁵, —OR⁶ and —NR⁶R⁶ are optionally substituted by 1 or 2 R⁹ groups, and wherein the alkyl moieties of said optional substituents are optionally substituted by halo or R⁹, with the proviso that two heteroatoms are not attached to the same carbon atom.

In some embodiments, each R¹ is independently selected from —NHSO₂R⁵, phthalimido-(C₁-C₄)-alkylsulfonylamino, benzamido, benzenesulfonylamino, 3-phenylureido, 2-oxopyrrolidin-1-yl, 2,5-dioxopyrrolidin-1-yl, and R¹⁰—(C₂-C₄)-alkanoylamino wherein R¹⁹ is selected from halo, —OR⁶, C₂-C₄ alkanoyloxy, —C(O)R⁷, and —NR⁶R⁶; and wherein said —NHSO₂R⁵, phthalimido-(C₁-C₄-alkylsulfonylamino, benzamido, benzenesulfonylamino, 3-phenylureido, 2-oxopyrrolidin-1-yl, 2,5-dioxopyrrolidin-1-yl, and R¹⁰—(C₂-C₄)-alkanoylamino R¹ groups are optionally substituted by 1 or 2 substituents independently selected from halo, C₁-C₄ alkyl, cyano, methanesulfonyl and C₁-C₄ alkoxy.

In some embodiments, two R¹ groups are taken together with the carbons to which they are attached to form a 5-15 membered ring that includes 1-6 heteroatoms selected from O, S, and N.

In some embodiments, R³ is hydrogen or C₁-C₆ alkyl optionally substituted by 1 to 3 substituents independently selected from halo, C₁-C₄ alkoxy, —NR⁶R⁶, and —SO₂R⁵.

In some embodiments, each R³ is independently selected from hydrogen, halo, hydroxy, C₁-C₆ alkyl, —NR⁶R⁶, and C₁-C₄ alkoxy, wherein the alkyl moieties of said R³ groups are optionally substituted by 1 to 3 substituents independently selected from halo, C₁-C₄ alkoxy, —NR⁶ R⁶, and —SO₂R⁵.

In some embodiments, at least one R¹ is hydrogen, at least three R³ are hydrogens, and at least one R³ is azido or -(ethynyl)-R¹¹, where R¹¹ is hydrogen or C₁-C₆ alkyl optionally substituted by hydroxy, —OR⁶, or —NR⁶R⁶.

In some embodiments, R^{1A}, R^{1D}, R², R^{3A}, R^{3B}, and R^{3E} are hydrogen; R^{3C} is hydrogen or halo; R^{3D} is halo or alkynyl; R^{1C} and R^{1D} are independently alkoxy, heteroarylalkoxy, or heterocyclylalkoxy or R^{1C} and R^{1D} are taken together with the carbons to which they are attached to form a 6-15 membered ring that includes 1-6 heteroatoms.

In some embodiments, R^{1A}, R^{1D}, R², R^{3A}, R^{3B}, and R^{3E} are hydrogen; R^{3C} is hydrogen or halo; R^{3D} is halo or lower alkynyl; R^{1C} and R^{1D} are (1-4C)alkoxy(1-4C)alkoxy, (2-4C)heterocyclyl(2-6C)alkoxy or R^{1C} and R^{1D} are taken together with the carbons to which they are attached to form a 6-15 membered ring that includes 1-6 oxygen heteroatoms.

In some embodiments, R^{1A}, R^{1D}, R², R^{3A}, R^{3B}, and R^{3E} are hydrogen; R^{3C} is hydrogen, fluorine or chlorine; R^{3D} is fluorine, chlorine, or H(2-4C)alkynyl; R^{1C} and R^{1D} are (1-4C)alkoxy(1-4C)alkoxy, (2-4C)heterocyclyl(2-6C)alkoxy or R^{1C} and R^{1D} are taken together with the carbons to which they are attached to form a 8-12 membered ring that includes 4-5 oxygen heteroatoms.

In some embodiments, R^{1A}, R^{1D}, R², R^{3A}, R^{3B}, and R^{3E} are hydrogen; R^{3C} is hydrogen, fluorine or chlorine; R^{3D} is fluorine, chlorine, or H(2C)alkynyl; R^{1C} and R^{1D} are (1-2C)alkoxy(2-4C)alkoxy, (4C)heterocyclyl(3-4C)alkoxy or R^{1C} and R^{1D} are taken together with the carbons to which they are attached to form a 8-12 membered ring that includes 4-5 oxygen heteroatoms.

One of skill in the art will recognize that the compositions and methods described herein include compounds related to erlotinib, gefitinb, and icotinib, including the compounds described in U.S. Pat. Nos. 5,457,105; 5,616,582; 5,770,599; 5,747,498; 6,900,221; 7,087,613; and RE41,065E, and U.S. Patent Publication No. 2011/01802882, which are each incorporated herein by reference in their entireties.

### DEFINITIONS

As used herein, the following terms are defined with the following meanings, unless expressly stated otherwise.

As used herein, the term “alkyl” refers to a branched or unbranched fully saturated acyclic aliphatic hydrocarbon group. An alkyl may be branched or straight chain. Alkyls may be substituted or unsubstituted. The alkyl group may have 1 to 20 carbon atoms (whenever it appears herein, a numerical range such as “1 to 20” or “1-20C” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 12 carbon atoms. The alkyl group could also be a lower alkyl having 1 to 6 carbon atoms. The alkyl group of the compounds may be designated as “C₄” or “C₁-C₄ alkyl” or “(1-4C)alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Alkyls include, but are not limited to, methyl, ethyl, propyl, isopropyl, butyl, isobutyl, tertiary butyl, pentyl, hexyl, and the like, each of which may be optionally substituted.

As used herein, the term “alkenyl” refers to a straight or branched chain aliphatic hydrocarbon of from two to twenty carbon atoms containing at least one carbon-carbon double bond including, but not limited to, 1-propenyl, 2-propenyl, 2-methyl-1-propenyl, 1-butenyl, 2-butenyl, and the like. In some embodiments, an alkenyl comprises 2 to 20 carbon atoms (whenever it appears herein, a numerical range such as “2 to 20” refers to each integer in the given range; e.g., “2 to 20 carbon atoms” means that an alkenyl group may comprise only 2 carbon atoms, 3 carbon atoms, etc., up to and including 20 carbon atoms, although the term “alkenyl” also includes instances where no numerical range of carbon atoms is designated). The alkenyl group may also be a medium size alkenyl having 2 to 12 carbon atoms. The alkenyl group could also be a lower alkenyl having 2 to 6 carbon atoms. An alkenyl may be designated as “C₂-C₆ alkenyl” or “(2-4C)alkenyl” or similar designations. By way of example only, “C₂-C₄ alkenyl” indicates an alkenyl having two, three, or four carbon atoms, e.g., the alkenyl is selected from ethenyl, propenyl, and butenyl.

As used herein, “alkynyl” refers to an alkyl group that contains in the straight or branched hydrocarbon chain one or more triple bonds. The alkynyl group may have 2 to 20 carbon atoms (whenever it appears herein, a numerical range such as “2 to 20” refers to each integer in the given range; e.g., “2 to 20 carbon atoms” means that the alkynyl group may consist of 2 carbon atom, 3 carbon atoms, 4 carbon atoms, etc., up to and including 20 carbon atoms, although the present definition also covers the occurrence of the term “alkynyl” where no numerical range is designated). The alkynyl group may also be a medium size alkynyl having 2 to 12 carbon atoms. The alkynyl group could also be a lower alkynyl having 2 to 6 carbon atoms. The alkynyl group of the compounds may be designated as “C₄” or “C₂-C₄ alkyl” or “(2-4C)alkynyl” or “H(2-4C)alkynyl” or similar designations. An alkynyl group may be unsubstituted or substituted.

As used herein, “cycloalkynyl” refers to a mono- or multi-cyclic hydrocarbon ring system that contains one or more triple bonds in at least one ring. If there is more than one triple bond, the triple bonds cannot form a fully delocalized pi-electron system throughout all the rings. When composed of two or more rings, the rings may be joined together in a fused fashion. A cycloalkynyl group may be unsubstituted or substituted.

As used herein, the term “alkoxy” refers to straight or branched chain alkyl covalently bonded to oxygen where the “alkoxy” is attached to the parent molecule through at least an oxygen linkage. Where an “alkoxy” substituent requires two points of attachment to the rest of the molecule the “alkoxy” is attached to the parent molecule through an oxygen linkage and a carbon linkage. Examples of alkoxy groups include, but are not limited to, methoxy, ethoxy, propoxy, isopropoxy, butoxy, n-butoxy, sec-butoxy, t-butoxy and the like. An alkoxy may be designated as “C₁-C₆ alkoxy” or similar designations. By way of example only, “C₁-C₄ alkoxy” indicates an alkyl having one, two, three, or four carbon atoms, e.g., the alkoxy is selected from methoxy, ethoxy, propoxy, iso-propoxy, butoxy, iso-butoxy, sec-butoxy, and tert-butoxy. The term alkoxy also includes substituents having multiple oxygen atoms, such as alkyloxyalkyloxy, alkoxyalkoxy, or (1-6C)alkoxy(1-6C)alkoxy. By way of example only, such substituents include methoxyethoxy, ethoxymethoxy, methoxypropoxy, and the like.

As used herein, the term “alkylthio” refers to straight or branched chain alkyl, an alkenyl, an alkynyl, a cycloalkyl, a cycloalkenyl or a cycloalkynylradical covalently bonded to the parent molecule through an —S— linkage. Examples of alkylthio groups include, but are not limited to, methanesulfide, ethanesulfide, propanesulfide, isopropanesulfide, butanesulfide, n-butanesulfide, sec-butanesulfide, tert-butanesulfide and the like.

As used herein, the term “amino” refers to a chemical moiety with formula —NHR′R″, where R′ and R″ are each independently selected from hydrogen, alkyl, cycloalkyl, aryl, heteroaryl (bonded through a ring carbon) and heteroalicyclic (bonded through a ring carbon).

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group (although the term “aryl” is defined as C₆-C₁₄ aryl when no numerical range is designated). Examples of aryl groups include, but are not limited to, phenyl, naphthyl, biphenyl, phenanthrenyl, naphthacenyl, and the like. In some embodiments, aryls may be substituted or unsubstituted.

As used herein, the term “cyano” refers to the group consisting of formula —CN.

As used herein, “cycloalkenyl” refers to a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). When composed of two or more rings, the rings may be connected together in a fused fashion. A cycloalkenyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s) or 3 to 8 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl.

As used herein, the term “halo” or “halogeno” refers to fluoro, chloro, bromo, or iodo.

As used herein, the term “heteroatom” refers to, for example, oxygen, sulfur and nitrogen.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

As used herein, “heterocycle” or “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline. As used herein, the term “oxo” or “carbonyl” refers to the group consisting of formula ═O or C═O.

Unless otherwise indicated, the term “optionally substituted” used herein refers to a group in which none, one, or more than one of the hydrogen atoms has been replaced with one or more group(s) individually and independently selected from: alkyl, alkenyl, cycloalkenyl, cycloalkyl, aryl, arylalkyl, heteroaryl, heterocyclyl, hydroxy, alkoxy, cyano, halo, oxo, thiocarbonyl, ester, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, and amino, including mono- and di-substituted amino groups, and the protected derivatives of amino groups. Such protective derivatives (and protecting groups that may form such protective derivatives) are known to those of skill in the art and may be found in references such as Greene and Wuts, above. When the group contains a nitrogen, or a sulfur, an oxo as a substituent also includes oxides, for example pyridine-N-oxide, thiopyran sulfoxide and thiopyran-S,S-dioxide. In embodiments in which two or more hydrogen atoms have been substituted, the substituent groups may together form a ring.

As used herein, the term “pharmaceutical agent” refers to a chemical compound or composition capable of inducing a desired therapeutic effect in a patient. In some embodiments, a pharmaceutical agent comprises an active agent, which is the agent that induces the desired therapeutic effect. In some embodiments, a pharmaceutical agent comprises a prodrug. In some embodiments, a pharmaceutical agent comprises inactive ingredients such as carriers, excipients, and the like.

Where the numbers of substituents is not specified (e.g. haloalkyl), there may be one or more substituents present. For example “haloalkyl” may include one or more of the same or different halogens.

As used herein, the term “therapeutically effective amount” refers to an amount of a pharmaceutical agent sufficient to achieve a desired therapeutic effect.

As used herein, the term “pharmaceutically acceptable” refers to a formulation of a compound that does not significantly abrogate the biological activity, a pharmacological activity and/or other properties of the compound when the formulated compound is administered to a patient. In some embodiments, a pharmaceutically acceptable formulation does not cause significant irritation to a patient.

As used herein, the term “pharmaceutically acceptable salt” refers to a formulation of a compound that does not cause significant irritation to an organism to which it is administered and does not abrogate the biological activity and properties of the compound. Pharmaceutical salts can be obtained by routine experimentation. Non-limiting examples of pharmaceutically acceptable salts include erlotinib HCl, gefitinib HCl, and icotinib HCl. Throughout the present disclosure, when a particular compound is mentioned by name, it is understood that the scope of the present disclosure encompasses pharmaceutically acceptable salts, esters, amides, or metabolites of the named compound.

As used herein, the term “co-administer” refers to administering more than one pharmaceutical agent to a patient. In some embodiments, co-administered pharmaceutical agents are administered together in a single dosage unit. In some embodiments, co-administered pharmaceutical agents are administered separately. In some embodiments, co-administered pharmaceutical agents are administered at the same time. In some embodiments, co-administered pharmaceutical agents are administered at different times.

As used herein, the term “patient” includes human and animal subjects.

As used herein, the term “substantially pure” means an object species (e.g., compound) is the predominant species present (i.e., on a molar basis it is more abundant than any other individual species in the composition). In some embodiments, a substantially purified fraction is a composition wherein the object species comprises at least about 50 percent (on a molar basis) of all species present. In some embodiments, a substantially pure composition will comprise more than about 80%, 85%, 90%, 95%, or 99% of all species present in the composition. In some embodiments, the object species is purified to essential homogeneity (contaminant species cannot be detected in the composition by conventional detection methods) wherein the composition consists essentially of a single species.

As used herein, the term “sustained release” has its ordinary meaning as understood by those skilled in the art and thus includes, by way of non-limiting example, the controlled release of a drug from a dosage form over an extended period of time. For example, in some embodiments, sustained-release dosage forms are those that have a release rate that is slower that of a comparable immediate release form, e.g., less than 80% of the release rate of an immediate-release dosage form.

As used herein, the term “oral dosage form” has its ordinary meaning as understood by those skilled in the art and thus includes, by way of non-limiting example, a formulation of a drug or drugs in a form administrable to a human, including pills, tablets, cores, capsules, caplets, loose powder, solutions, and suspensions.

Throughout the specification, groups and substituents thereof can be chosen by one skilled in the field to provide stable moieties and compounds.

### Methods of Preparation

Some embodiments described herein provide a composition comprising a pharmaceutically acceptable excipient, and a compound of any of the embodiments described herein.

In some embodiments, at least one compound described herein, or pharmaceutically acceptable salt, ester, amide, and/or prodrug thereof, either alone or combined with one or more pharmaceutically acceptable carriers, forms a pharmaceutical agent. Techniques for formulation and administration of the compounds described herein may be found for example, in “Remington's Pharmaceutical Sciences,” Mack Publishing Co., Easton, Pa., 18th edition, 1990, which is incorporated herein by reference in its entirety.

In some embodiments, a pharmaceutical agent comprising one or more compounds described herein is prepared using known techniques, including, but not limited to mixing, dissolving, granulating, dragee-making, levigating, emulsifying, encapsulating, entrapping or tabletting processes.

In some embodiments, a pharmaceutical agent comprising one or more compounds described herein is a liquid (e.g., a suspension, elixir and/or solution). In certain of such embodiments, a liquid pharmaceutical agent comprising one or more compounds described herein is prepared using ingredients known in the art, including, but not limited to, water, glycols, oils, alcohols, flavoring agents, preservatives, and coloring agents.

In some embodiments, a pharmaceutical agent comprising one or more compounds described herein is a solid (e.g., a powder, tablet, and/or capsule). In certain of such embodiments, a solid pharmaceutical agent comprising one or more compounds described herein is prepared using ingredients known in the art, including, but not limited to, starches, sugars, diluents, granulating agents, lubricants, binders, and disintegrating agents.

In some embodiments, a pharmaceutical agent comprising one or more compounds described herein is formulated as a depot preparation. Certain such depot preparations are typically longer acting than non-depot preparations. In some embodiments, such preparations are administered by implantation (for example subcutaneously or intramuscularly) or by intramuscular injection. In some embodiments, depot preparations are prepared using suitable polymeric or hydrophobic materials (for example an emulsion in an acceptable oil) or ion exchange resins, or as sparingly soluble derivatives, for example, as a sparingly soluble salt.

In some embodiments, a pharmaceutical agent comprising one or more compounds described herein comprises a delivery system. Examples of delivery systems include, but are not limited to, liposomes and emulsions. Certain delivery systems are useful for preparing certain pharmaceutical agents including those comprising hydrophobic compounds. In some embodiments, certain organic solvents such as dimethylsulfoxide are used.

In some embodiments, a pharmaceutical agent comprising one or more compounds described herein comprises one or more tissue-specific delivery molecules designed to deliver the pharmaceutical agent to specific tissues or cell types. For example, in some embodiments, pharmaceutical agents include liposomes coated with a tissue-specific antibody.

In some embodiments, a pharmaceutical agent comprising one or more compounds described herein comprises a co-solvent system. Certain of such co-solvent systems comprise, for example, benzyl alcohol, a nonpolar surfactant, a water-miscible organic polymer, and an aqueous phase. In some embodiments, such co-solvent systems are used for hydrophobic compounds. The proportions of such co-solvent systems may be varied considerably without significantly altering their solubility and toxicity characteristics.

In some embodiments, a pharmaceutical agent comprising one or more compounds described herein comprises a sustained-release system. A non-limiting example of such a sustained-release system is a semi-permeable matrix of solid hydrophobic polymers. In some embodiments, sustained-release systems may, depending on their chemical nature, release compounds over a period of hours, days, weeks or months.

Certain compounds used in a pharmaceutical agent described herein may be provided as pharmaceutically acceptable salts with pharmaceutically compatible counterions. Pharmaceutically compatible salts may be formed with many acids, including but not limited to hydrochloric, sulfuric, acetic, lactic, tartaric, malic, or succinic.

### Methods of Screening

Provided herein are methods of screening compounds for the treatment of a condition described herein. In some embodiments, the methods comprise the inhibition of TNF-α. In some embodiments, the methods comprise the inhibition of EGFR. Methods of screening can include methods of evaluating the inhibitory properties of a compound, such as, but not limited to, a member of the set of compounds provided herein. In some embodiments, the methods comprise contacting TNF-α with a compound, and evaluating the level of TNF-α inhibition. In some embodiments, the methods comprise contacting EGFR with a compound, and evaluating the level of EGFR inhibition. In some embodiments, the methods comprise contacting a cell with a compound, and evaluating the level of cellular proliferation. In some embodiments, the compound is a compound as disclosed herein.

In some embodiments, the methods can comprise evaluating the level of TNF-α inhibition using a cell-free assay as described herein or otherwise known in the art. Inhibition can be determined by whether TNF-α activity is reduced according to a user-selected level, as described herein or otherwise known in the art. Thus, in some embodiments, a user-selected level of inhibition can be, for example, at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% inhibition of TNF-α activity.

In some embodiments, the methods can comprise evaluating the level of EGFR inhibition using a cell-free assay as described herein or otherwise known in the art. Inhibition can be determined by whether EGFR activity is reduced according to a user-selected level, as described herein or otherwise known in the art. Thus, in some embodiments, a user-selected level of inhibition can be, for example, at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% inhibition of EGFR activity.

The results of the methods of evaluating the inhibitory properties of the compounds provided herein can be reported in terms understood in the art including, for example, IC₅₀ or EC₅₀, or other standard terms known in the art. Thus, the evaluation methods provided herein can include evaluating the results where evaluating the results includes determining the inhibitory properties of the compound(s) being tested. In some embodiments evaluating the results also includes comparing the inhibitory properties of a compound being tested to the inhibitory properties of one or more reference compounds.

In some embodiments, the methods can comprise an in vitro assay on whole cells as described herein or otherwise known in the art. For example, in some embodiments, the method comprises measuring inhibition of TNF-α in cells involved in an inflammatory process. In some embodiments, the method comprises measuring inhibition of EFGR in cells involved in an inflammatory process.

In some embodiments, the methods can comprise an in vivo assay as described herein or otherwise known in the art. For example, in some embodiments, the method comprises treating an animal with a compound provided herein, and evaluating the effects of treating the animal with the compound.

### Methods of Administration

In some embodiments, a pharmaceutical agent comprising one or more compounds described herein comprises an active ingredient in a therapeutically effective amount. In some embodiments, the therapeutically effective amount is sufficient to prevent, alleviate, or ameliorate symptoms of a disease or to prolong the survival of the subject being treated. Determination of a therapeutically effective amount is within the capability of those skilled in the art.

In some embodiments, a pharmaceutical agent comprising one or more compounds described herein is useful for treating a condition or disorder in a mammal, and particularly in a human patient. Suitable administration routes include, but are not limited to, oral, rectal, transmucosal, intestinal, enteral, topical, suppository, through inhalation, intrathecal, intraventricular, intraperitoneal, intranasal, intraocular and parenteral (e.g., intravenous, intramuscular, intramedullary, and subcutaneous). In some embodiments, the compositions described herein are administered to achieve local rather than systemic exposures. For example, pharmaceutical agents may be injected directly in the area of desired effect (e.g., in a synovial joint). In some preferred embodiments, the compositions described herein are in oral dosage form.

In some embodiments, a pharmaceutical agent comprising a compound described herein is prepared for oral administration. In some embodiments, a pharmaceutical agent is formulated by combining one or more compounds described herein with one or more pharmaceutically acceptable carriers. In some embodiments, carriers enable the compounds described herein to be formulated as tablets, pills, core, dragees, capsules, liquids, gels, syrups, slurries, suspensions and the like, for oral ingestion by a patient. In a preferred embodiment, the oral dosage form is in the form of a pill, tablet, or capsule. In some embodiments, pharmaceutical agents for oral use are obtained by mixing one or more compounds described herein and one or more solid excipient. Suitable excipients include, but are not limited to, fillers, such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In some embodiments, such a mixture is optionally ground and auxiliaries are optionally added. In some embodiments, pharmaceutical agents are formed to obtain tablets or dragee cores. In some embodiments, disintegrating agents (e.g., cross-linked polyvinyl pyrrolidone, agar, or alginic acid or a salt thereof, such as sodium alginate) are added.

The amount of active ingredient that may be combined with carrier materials to produce a dosage form can vary depending on the patient being treated and the particular mode of administration. A typical pharmaceutical preparation can contain from about 5% to about 95% active ingredient (w/w). In other embodiments, the pharmaceutical preparation can contain from about 20% to about 80% active ingredient.

In some embodiments, dragee cores are provided with coatings. In some embodiments, concentrated sugar solutions are used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to tablets or dragee coatings.

In some embodiments, pharmaceutical agents for oral administration are push-fit capsules made of gelatin. In some embodiments, push-fit capsules comprise one or more compounds described herein in admixture with one or more filler such as lactose, binders such as starches, and/or lubricants such as talc or magnesium stearate and, optionally, stabilizers. In some embodiments, pharmaceutical agents for oral administration are soft, sealed capsules made of gelatin and a plasticizer, such as glycerol or sorbitol. In certain soft capsules, one or more compounds described herein are be dissolved or suspended in suitable liquids, such as fatty oils, liquid paraffin, or liquid polyethylene glycols. In addition, stabilizers may be added.

In some embodiments, a pharmaceutical agent is in powder form for constitution with a suitable vehicle, e.g., sterile pyrogen-free water, before use.

In some embodiments, pharmaceutical agents are prepared for buccal administration. Certain of such pharmaceutical agents are tablets or lozenges formulated in conventional manner.

In some embodiments, a pharmaceutical agent is prepared for administration by injection (e.g., intravenous, subcutaneous, intramuscular, etc.). In some embodiments, a pharmaceutical agent comprises a carrier and is formulated in aqueous solution, such as water or physiologically compatible buffers such as Hanks's solution, Ringer's solution, or physiological saline buffer. In some embodiments, other ingredients are included (e.g., ingredients that aid in solubility or serve as preservatives). In some embodiments, injectable suspensions are prepared using appropriate liquid carriers, suspending agents and the like. Certain pharmaceutical agents for injection are presented in unit dosage form, e.g., in ampoules or in multi-dose containers. Certain pharmaceutical agents for injection are suspensions, solutions or emulsions in oily or aqueous vehicles, and may contain formulatory agents such as suspending, stabilizing and/or dispersing agents. Certain solvents suitable for use in pharmaceutical agents for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the compounds to allow for the preparation of highly concentrated solutions.

In some embodiments, a pharmaceutical agent is prepared for transmucosal administration. In certain of such embodiments penetrants appropriate to the barrier to be permeated are used in the formulation. Such penetrants are generally known in the art.

In some embodiments, a pharmaceutical agent is prepared for administration by inhalation. In some embodiments, pharmaceutical agents for inhalation are prepared in the form of an aerosol spray in a pressurized pack or a nebulizer. In some embodiments, pharmaceutical agents comprise a propellant, e.g., dichlorodifluoromethane, trichlorofluoromethane, dichlorotetrafluoroethane, carbon dioxide or other suitable gas. In some embodiments using a pressurized aerosol, the dosage unit may be determined with a valve that delivers a metered amount. In some embodiments, capsules and cartridges for use in an inhaler or insufflator may be formulated. Certain formulations comprise a powder mixture of a compound described herein and a suitable powder base such as lactose or starch.

In some embodiments, a pharmaceutical agent is prepared for rectal administration, such as a suppositories or retention enema. In some embodiments, pharmaceutical agents comprise known ingredients, such as cocoa butter and/or other glycerides.

In some embodiments, a pharmaceutical agent is prepared for topical administration. Certain of such pharmaceutical agents comprise bland moisturizing bases, such as ointments or creams. Exemplary suitable ointment bases include, but are not limited to, petrolatum, petrolatum plus volatile silicones, lanolin and water in oil emulsions.

In some embodiments in which a pharmaceutical agent is administered locally, the dosage regimen is adjusted to achieve a desired local concentration of a compound described herein.

The dosage of the compositions described herein can range from about 0.01 mg to about 100 mg/kg patient body weight per day, in 1 to 5 divided doses per day. In some embodiments, the compounds as described herein can be administered at a dosage of about 0.5 mg to about 75 mg/kg patient body weight per day, in 1 to 5 divided doses per day. However, those of skill will readily appreciate that dose levels can vary as a function of the specific compound, the severity of the symptoms and the susceptibility of the subject to side effects. Preferred dosages for a given compound as described herein can be readily determinable by those of skill in the art by a variety of means.

In some embodiments, a pharmaceutical agent described herein is administered to a patient between about 0.1% and 500%, 5% and 200%, 10% and 100%, 15% and 85%, 25% and 75%, or 40% and 60% of an established human dosage. Where no human dosage is established, a suitable human dosage may be inferred from ED₅₀ or ID₅₀ values, or other appropriate values derived from in vitro or in vivo studies.

In some embodiments, a daily dosage regimen for a patient comprises an oral dose of between 0.1 mg and 2000 mg, 5 mg and 1500 mg, 10 mg and 1000 mg, 20 mg and 500 mg, 30 mg and 200 mg, or 40 mg and 100 mg of a compound described herein. In some embodiments, a daily dosage regimen is administered as a single daily dose. In some embodiments, a daily dosage regimen is administered as two, three, four, or more than four doses.

In some embodiments, a pharmaceutical agent comprising one or more compounds described herein is administered in the form of a dosage unit (e.g., a tablet, capsule, bolus, etc.). In some embodiments, such dosage units comprise a compound as disclosed and described herein in a dose from about 1 μg/kg of body weight to about 50 mg/kg of body weight. In some embodiments, such dosage units comprise a compound as disclosed and described herein in a dose from about 2 μg/kg of body weight to about 25 mg/kg of body weight. In some embodiments, such dosage units comprise a compound as disclosed and described herein in a dose from about 10 μg/kg of body weight to about 5 mg/kg of body weight. In some embodiments, pharmaceutical agents are administered as needed, once per day, twice per day, three times per day, or four or more times per day. It is recognized by those skilled in the art that the particular dose, frequency, and duration of administration depends on a number of factors, including, without limitation, the biological activity desired, the condition of the patient, and tolerance for the pharmaceutical agent.

In some embodiments, a pharmaceutical agent is administered as a single dose. In some embodiments, multiple doses of a composition are administered. For example, in some embodiments, a composition is administered once per month, twice per month, three times per month, every other week (QOW), once per week (QW), twice per week (BIW), three times per week (TIW), four times per week, five times per week, six times per week, every other day (QOD), daily (QD), twice a day (QID), or three times a day (TID), over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

In some embodiments, the administration of a compound described herein is continued for a period of, or of about, 1, 2, 3, 4, 6, 8, 10, 12, 16, 20, 24, 36, 48, or 52 weeks, or a range defined by any two of the preceding values. In some embodiments, the administration of a compound described herein is continued until the reduction in symptoms of a disease, disorder, or condition is stabilized for a period of, or of about, 1, 2, 3, 4, 5, 6, or more weeks, or a range defined by any two of the preceding values. For example, in some embodiments, the administration of a compound described herein is continued until the condition is stabilized for a period of, or of about, 1, 2, 3, 4, 5, 6, or more weeks, or a range defined by any two of the preceding values. In some embodiments, administration of a compound described herein is continued until the individual no longer needs a treatment.

In some embodiments, a compound described herein is administered once per day. In some embodiments, a compound described herein is administered two, three, four, or more times per day. A compound described herein can be administered less than once per day, for example once every 2, 3, 4, 5, 6, 7, 8, 9, 10, 11, 12, 13, or 14 days, or every 1 or 2 weeks, or a range defined by any two of the preceding values. In some embodiments, the number of administrations per day is constant (e.g., one time per day). In other embodiments, the number of administrations is variable. In some embodiments, a compound described herein is divided into equal doses and administered more than once per day. In some embodiments, a compound described herein is divided into unequal doses and administered more than once per day. In some embodiments, a compound described herein is divided into a different number of doses and is administered a different number of times per day. However, the number of administrations may change depending on effectiveness of the dosage form, observed side effects, external factors (e.g., a change in another medication), or the length of time that the dosage form has been administered.

In some embodiments, a compound described herein is administered with varying frequency during treatment. In some of these embodiments, the varying frequency comprises a decreased frequency over time. For example, a compound described herein can be initially administered more than once per day, followed by administration only once per day at a later point in treatment. In some embodiments, the daily dosage of a compound described herein is consistent despite the varying frequency of administration. For example, in some embodiments, two tablets of a compound described herein are initially administered twice per day, while four tablets of a compound described herein are administered once per day at a later point in treatment.

In some embodiments, a compound described herein is administered in consistent daily dosages throughout the period of treatment. In some embodiments, a compound described herein is administered in varying daily dosages during the period of treatment. In some of these embodiments, the daily dosages comprise increasing daily dosages over time. In some of these embodiments, the daily dosages comprise decreasing daily dosages over time.

In some embodiments where a compound described herein is administered less than once per day in a controlled release (CR) or sustained release (SR) formulation, the dose is selected so that the patient receives a daily dose that is about the same as a daily dose described herein.

In some embodiments, a pharmaceutical agent described herein is administered for a period of continuous therapy. For example, a pharmaceutical agent described herein may be administered over a period of days, weeks, months, or years.

Dosage amount, interval between doses, and duration of treatment may be adjusted to achieve a desired effect. In some embodiments, dosage amount and interval between doses are adjusted to maintain a desired concentration on compound in a patient. For example, in some embodiments, dosage amount and interval between doses are adjusted to provide plasma concentration of a compound described herein at an amount sufficient to achieve a desired effect. In certain of such embodiments the plasma concentration is maintained above the minimal effective concentration (MEC). In some embodiments, pharmaceutical agents described herein are administered with a dosage regimen designed to maintain a concentration above the MEC for about 10-90% of the time, between about 30-90% of the time, or between about 50-90% of the time.

In some embodiments, a pharmaceutical agent described herein is administered by continuous intravenous infusion. In certain of such embodiments, from 0.1 mg to 500 mg of a composition of described herein is administered per day.

In some embodiments, the daily dose of erlotinib can range from about 25 mg to about 1000 mg, or about 25 mg to about 500 mg, or about 50 mg to about 250 mg, or about 100 mg to about 150 mg. In some embodiments, the daily dose is about 25 mg to about 1000 mg, or about 25 mg to about 500 mg, or about 50 mg to about 250 mg, or about 100 mg to about 150 mg of erlotinib, or a range defined by any two of the preceding values. In some embodiments, the daily dose is about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 600 mg, about 700 mg, about 800 mg, about 900 mg, or about 1000 mg. In a preferred embodiment, the daily dose is about 25, 100, or 150 mg, or a range defined by any two of the preceding values. The selection of a particular dosage may be based on the weight of the patient. The selection of a particular dosage may be based on the identity, dosage, and/or dosing schedule of another co-administered compound. However, in some embodiments, it may be necessary to use dosages outside these ranges. In some embodiments, the daily dose is administered in a single oral dosage form. In some embodiments, the daily dose of erlotinib is the same, and in some embodiments, the daily dose is different.

In some embodiments, the daily dose of gefitinib can range from about 25 mg to about 1000 mg, or about 25 mg to about 500 mg, or about 50 mg to about 250 mg, or about 100 mg to about 150 mg. In some embodiments, the daily dose is about 25 mg to about 1000 mg, or about 25 mg to about 500 mg, or about 50 mg to about 250 mg, or about 100 mg to about 150 mg of gefitinib, or a range defined by any two of the preceding values. In some embodiments, the daily dose is about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 600 mg, about 700 mg, about 800 mg, about 900 mg, or about 1000 mg. In a preferred embodiment, the daily dose is about 250 mg. The selection of a particular dosage may be based on the weight of the patient. The selection of a particular dosage may be based on the identity, dosage, and/or dosing schedule of another co-administered compound. However, in some embodiments, it may be necessary to use dosages outside these ranges. In some embodiments, the daily dose is administered in a single oral dosage form. In some embodiments, the daily dose of gefitinib is the same, and in some embodiments, the daily dose is different.

In some embodiments, the daily dose of icotinib can range from about 25 mg to about 1000 mg, or about 25 mg to about 500 mg, or about 50 mg to about 250 mg, or about 100 mg to about 150 mg. In some embodiments, the daily dose is about 25 mg to about 1000 mg, or about 25 mg to about 500 mg, or about 50 mg to about 250 mg, or about 100 mg to about 150 mg of icotinib, or a range defined by any two of the preceding values. In some embodiments, the daily dose is about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 600 mg, about 700 mg, about 800 mg, about 900 mg, or about 1000 mg. In a preferred embodiment, the daily dose is about 300 to 750 mg. The selection of a particular dosage may be based on the weight of the patient. The selection of a particular dosage may be based on the identity, dosage, and/or dosing schedule of another co-administered compound. However, in some embodiments, it may be necessary to use dosages outside these ranges. In some embodiments, the daily dose is administered in a single oral dosage form. In some embodiments, the daily dose of icotinib is the same, and in some embodiments, the daily dose is different.

In some embodiments, the act of providing includes supplying, acquiring, or administering a composition described herein. In some embodiments, “administering” a drug includes an individual obtaining and taking a drug on their own. For example, in some embodiments, an individual obtains a drug from a pharmacy and self-administers the drug in accordance with the methods provided herein.

In some embodiments, the present invention relates to a kit. The kit may include one or more unit dosage forms comprising a compound described herein. The unit dosage forms may be of an oral formulation. For example, the unit dosage forms may comprise pills, tablets, or capsules. The kit may include a plurality of unit dosage forms. In some embodiments, the unit dosage forms are in a container.

The methods, compositions and kits disclosed herein may include information. The information may be in a form prescribed by a governmental agency regulating the manufacture, use, or sale of pharmaceuticals, which notice is reflective of approval by the agency of the form of the drug for human or veterinary administration. Such information, for example, may be the labeling approved by the U.S. Food and Drug Administration for prescription drugs, or the approved product insert. The information can include required information regarding dose and dosage forms, administration schedules and routes of administration, adverse events, contraindications, warning and precautions, drug interactions, and use in specific populations (see, e.g., 21 C.F.R. §201.57 which is incorporated herein by reference in its entirety), and in some embodiments is required to be present on or associated with the drug for sale of the drug. Dosage forms comprising a compound described herein formulated in a compatible pharmaceutical carrier may also be prepared, placed in an appropriate container, and labeled for treatment of an indicated condition. In some embodiments, a kit is for sale of a prescription drug requiring the approval of and subject to the regulations of a governmental agency, such as the Food and Drug Administration of the United States. In some embodiments, the kit comprises the label or product insert required by the agency, such as the FDA, for sale of the kit to consumers, for example in the U.S.

In some embodiments, a pharmaceutical agent may be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound described herein formulated in a compatible pharmaceutical carrier may also be prepared, placed in an appropriate container, and labeled for treatment of an indicated condition.

The information may comprise instructions to administer the unit dosage form at a dosage of about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 600 mg, about 700 mg, about 800 mg, about 900 mg, or about 1000 mg of erlotinib or a pharmaceutically acceptable salt thereof. The information may comprise instructions to administer the unit dosage form at a dosage of about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 600 mg, about 700 mg, about 800 mg, about 900 mg, or about 1000 mg of gefitinib or a pharmaceutically acceptable salt thereof. The information may comprise instructions to administer the unit dosage form at a dosage of about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 600 mg, about 700 mg, about 800 mg, about 900 mg, or about 1000 mg of icotinib or a pharmaceutically acceptable salt thereof. These instructions may be provided in a variety of ways.

The information may comprise instructions about when to administer the unit dosage forms. For example, the information may comprise instructions about when to administer the unit dosage forms relative to the administration of another medication or food. In preferred embodiments, the information instructs an individual to take a composition with food. In preferred embodiments, the information instructs an individual to take a composition without food. In some embodiments, the compositions described herein are administered with food. In some embodiments, the compositions described herein are administered without food. For example, in some embodiments, a composition is administered no less than one or two hours following a meal.

Instructions and/or information may be present in a variety of forms, including printed information on a suitable medium or substrate (e.g., a piece or pieces of paper on which the information is printed), computer readable medium (e.g., diskette, CD, etc. on which the information has been recorded), or a website address that may be accessed via the internet. Printed information may, for example, be provided on a label associated with a drug product, on the container for a drug product, packaged with a drug product, or separately given to the patient apart from a drug product, or provided in manner that the patient can independently obtain the information (e.g., a website). Printed information may also be provided to a medical caregiver involved in treatment of the patient. In some embodiments, the information is provided to a person orally.

Some embodiments comprise a therapeutic package suitable for commercial sale. Some embodiments comprise a container. The container can be in any conventional shape or form as known in the art which is made of a pharmaceutically acceptable material, for example a paper or cardboard box, a glass or plastic bottle or jar, a re-sealable bag (e.g., to hold a “refill” of tablets for placement into a different container), or a blister pack with individual dosages for pressing out of the pack according to a therapeutic schedule. The container employed can depend on the exact dosage form involved, e.g., a conventional cardboard box would not generally be used to hold a liquid suspension. It is feasible that more than one container can be used together in a single package to market a single dosage form. For example, tablets may be contained in a bottle which is in turn contained within a box.

The information can be associated with the container, for example, by being: written on a label (e.g., the prescription label or a separate label) adhesively affixed to a bottle containing a dosage form described herein; included inside a container as a written package insert, such as inside a box which contains unit dose packets; applied directly to the container such as being printed on the wall of a box; or attached as by being tied or taped, e.g., as an instructional card affixed to the neck of a bottle via a string, cord or other line, lanyard or tether type device. The information may be printed directly on a unit dose pack or blister pack or blister card.

### Indications

Based on the teachings provided herein, the compounds provided herein can be used in methods of treating or relieving the symptoms of a condition described herein. In a preferred embodiment, a composition described herein is used in the treatment of an inflammatory condition. In some embodiments, the inflammatory condition is an autoimmune condition.

In some embodiments, the inflammatory condition is type 2 diabetes. In some embodiments, the compositions described herein are useful for reducing glycated hemoglobin (HbAlc) levels. In some embodiments, the HbAlc level is reduced by about 1%, 2%, 3%, 4%, 5%, 6%, 7%, 8%, 9%, 10%, 15%, 20%, 25%, 30%, 35%, 40%, 45%, or 50%. In some embodiments, the HbAlc level is reduced to about 7.0%, 6.9%, 6.8%, 6.7%, 6.6%, 6.5%, 6.4%, 6.3%, 6.2%, 6.1%, 6.0%, 5.9%, 5.8%, 5.7%, 5.6%, 5.5%, 5.4%, 5.3%, 5.2%, 5.1%, 5.0%, 4.9%, 4.8%, 4.7%, 4.6%, 4.5%, 4.4%, 4.3%, 4.2%, 4.1%, or 4.0%. In some embodiments, the HbAlc level is reduced to about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mmol/mol. In some embodiments, the inflammatory condition is an autoimmune disease. In some embodiments, the autoimmune disease is selected from the group consisting of acute disseminated encephalomyelitis (ADEM), acute necrotizing hemorrhagic leukoencephalitis, Addison's disease, agammaglobulinemia, alopecia areata, amyloidosis, ankylosing spondylitis, anti-GBM/Anti-TBM nephritis, antiphospholipid syndrome (APS), autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune pancreatitis, autoimmune retinopathy, autoimmune thrombocytopenic purpura (ATP), autoimmune thyroid disease, autoimmune urticaria, axonal & neuronal neuropathies, Balo disease, Behcet's disease, bullous pemphigoid, cardiomyopathy, Castleman disease, celiac disease, Chagas disease, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogans syndrome, cold agglutinin disease, congenital heart block, coxsackie myocarditis, CREST disease, essential mixed cryoglobulinemia, demyelinating neuropathies, dermatitis herpetiformis, dermatomyositis, discoid lupus, Dressler's syndrome, endometriosis, eosinophilic fasciitis, erythema nodosum, experimental allergic encephalomyelitis, Evans syndrome, fibrosing alveolitis, giant cell arteritis (temporal arteritis), glomerulonephritis, Goodpasture's syndrome, granulomatosis with polyangiitis (GPA) (also known as Wegener's granulomatosis), Graves' disease, Guillain-Barre syndrome, Hashimoto's encephalopathy, Hashimoto's thyroiditis, hemolytic anemia, Henoch-Schonlein purpura, herpes gestationis, hypogammaglobulinemia, idiopathic thrombocytopenic purpura (ITP), IgA nephropathy, IgG4-related sclerosing disease, immunoregulatory lipoproteins, inclusion body myositis, insulin-dependent diabetes (also known as type 1 diabetes), interstitial cystitis, juvenile arthritis, juvenile diabetes, Kawasaki syndrome, Lambert-Eaton syndrome, leukocytoclastic vasculitis, lichen planus, lichen sclerosus, ligneous conjunctivitis, linear IgA disease (LAD), chronic Lyme disease, Meniere's disease, microscopic polyangiitis, mixed connective tissue disease (MCTD), Mooren's ulcer, Mucha-Habermann disease, multiple sclerosis, myasthenia gravis, myositis, narcolepsy, neuromyelitis optica (also known as Devic's disease or Devic's syndrome), neutropenia, ocular cicatricial pemphigoid, optic neuritis, palindromic rheumatism, Pediatric Autoimmune Neuropsychiatric Disorders Associated with Streptococcus (PANDAS) paraneoplastic cerebellar degeneration, paroxysmal nocturnal hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, autoimmune polyglandular syndromes (type I, II, & III), polymyalgia rheumatica, polymyositis, postmyocardial infarction syndrome, postpericardiotomy syndrome, progesterone dermatitis, primary biliary cirrhosis, primary sclerosing cholangitis. psoriasis, psoriatic arthritis, idiopathic pulmonary fibrosis, pyoderma gangrenosum, pure red cell aplasia, Raynauds phenomenon, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm & testicular autoimmunity, stiff person syndrome, subacute bacterial endocarditis (SBE), Susac's syndrome, sympathetic ophthalmia, systemic lupus erythematosus (SLE), Takayasu's arteritis, temporal arteritis/giant cell arteritis, thrombocytopenic purpura (TTP), Tolosa-Hunt syndrome, transverse myelitis, ulcerative colitis, undifferentiated connective tissue disease (UCTD), uveitis, vasculitis, vesiculobullous dermatosis, and vitiligo. In some embodiments, the autoimmune disease is a disorder in which the inhibition of a TNF-alpha inhibitor has demonstrated therapeutic efficacy. In a preferred embodiment, the autoimmune disease is selected from the group consisting of rheumatoid arthritis, psoriasis, and Crohn's disease. In another preferred embodiment, the autoimmune disease is type 2 diabetes. In another preferred embodiment, the autoimmune disease is multiple sclerosis. In some embodiments, the multiple sclerosis is relapsing remitting multiple sclerosis. In some embodiments, the multiple sclerosis is secondary progressive multiple sclerosis. In some embodiments, the multiple sclerosis is primary progressive multiple sclerosis. In some embodiments, the multiple sclerosis is progressive relapsing multiple sclerosis. In some embodiments, the multiple sclerosis is recurring remitting multiple sclerosis. In some embodiments, the compositions described herein are used for the treatment of multiple sclerosis. In some embodiments, the compositions described herein are used for the treatment of psoriasis. In some embodiments, the compositions described herein are used for the treatment of inflammatory bowel disease. In some embodiments, the compositions described herein are used for the treatment of Crohn's disease. In some embodiments, the compositions described herein are used for the treatment of rheumatoid arthritis. In some embodiments, the compositions described herein are used for the treatment of ulcerative colitis. In some embodiments, the compositions described herein are used for the treatment of Hashimoto's thyroiditis.

In some embodiments, the formulation, route of administration, and dosage for a pharmaceutical agent described herein can be chosen in view of a particular patient's condition, for which guidance can be found in “The Pharmacological Basis of Therapeutics,” Fingl et al., 1975, which is incorporated herein by reference in its entirety.

In any of the embodiments described herein, methods of treatment can alternatively entail use claims, such as second medical use claims. For example, a method of treating Crohns' disease with a composition can alternatively entail the use of a composition in the manufacture of a medicament for the treatment of Crohns' disease, or the use of a composition for the treatment of Crohns' disease.

It will be understood by those of skill in the art that numerous and various modifications can be made without departing from the spirit of the present invention. Therefore, it should be clearly understood that the embodiments disclosed herein are illustrative only and are not intended to limit the scope of the present invention. The use of the singular includes the plural unless specifically stated otherwise. The use of “or” means “and/or” unless stated otherwise. The use of the term “including,” as well as other forms, such as “includes,” and “included,” is not limiting. Further, the section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

### Example 1

A 73 year old male, whose daughter has a history of recurrent-remitting MS, developed severe back pain. Despite never having smoked, he was found to have metastatic NSCLC with a genetic mutation. He also had a greater than ten year history of mild plaque psoriasis on both elbows that was unresponsive to topical treatment, a history of type 2 insulin requiring diabetes mellitus associated with metabolic syndrome and insulin resistance, and Hashimoto's thyroiditis. Three weeks after starting 150 mg of daily oral erlotinib, his pain abated and his psoriasis cleared. After ten weeks, he no longer required insulin. After seven months, there was no evidence of NSCLC or psoriasis, and he remains off of insulin and has not required any new medication to control his diabetes. After one year, his serum level of antithyroid peroxidase was reduced from 1719 to 366.

### Example 2

A 65 year old female was well until age 41, when she developed numbness and tingling in her lower extremities. A magnetic resonance imaging (MRI) of the brain, along with spinal fluid findings, were consistent with MS. She was treated with intravenous methyl prednisone and achieved total remission. From then until age 61, she was asymptomatic except for three exacerbations which also responded to intravenous methyl prednisone. She took no other medications for MS.

At age 61, while still asymptomatic, a routine MRI showed areas of demyelination in her spinal cord. She was placed on interferon β-1a. At age 63, she developed weakness in her lower extremities and was switched to natalizumab. She has been asymptomatic since that time. At age 65, independent of her MS, she developed NSCLC with a genetic mutation. Natalizumab was discontinued and erlotinib was initiated at an oral dose of 150 mg daily. After six months, her cancer is in total remission. Brain MRIs with contrast two months prior to starting erlotinib, immediately prior to starting erlotinib, and after three and six months on erlotinib are unchanged. There are no enhancing lesions, nor are there any changes in her several small pre-existing plaques.

### Example 3

A patient is diagnosed with rheumatoid arthritis. Erlotinib is administered in a dose of 150 mg daily for several weeks. Clinical improvement is seen in the patient. Following clinical improvement, erlotinib is reduced to a maintenance dose as low as 25 mg daily.

### Example 4

A patient is diagnosed with Crohn's disease. Erlotinib is administered in a dose of 150 mg daily for several weeks. Clinical improvement is seen in the patient. Following clinical improvement, erlotinib is reduced to a maintenance dose as low as 25 mg daily.

### Example 5

A group of patients with multiple sclerosis is identified. The patients are administered erlotinib (150 mg daily), gefitinib (250 mg daily), icotinib (300 mg daily), or placebo in for several weeks. Clinical improvement is seen in the patients administered erlotinib, gefitinib, and icotinib, but not in the patients administered placebo. Following clinical improvement, the dose of erlotinib, gefitinb, or icotinib is reduced to a maintenance dose as low as 25 mg, 50 mg, or 75 mg daily, respectively.

### Example 6

A group of patients with insulin resistance is identified. The patients are administered erlotinib (150 mg daily), gefitinib (250 mg daily), icotinib (300 mg daily), or placebo in for several weeks. Clinical improvement is seen in the patients administered erlotinib, gefitinib, and icotinib, but not in the patients administered placebo. Following clinical improvement, the dose of erlotinib, gefitinb, or icotinib is reduced to a maintenance dose as low as 25 mg, 50 mg, or 75 mg daily, respectively.

### Example 7

A group of patients with psoriasis is identified. The patients are administered erlotinib (150 mg daily), gefitinib (250 mg daily), icotinib (300 mg daily), or placebo in for several weeks. Clinical improvement is seen in the patients administered erlotinib, gefitinib, and icotinib, but not in the patients administered placebo. Following clinical improvement, the dose of erlotinib, gefitinb, or icotinib is reduced to a maintenance dose as low as 25 mg, 50 mg, or 75 mg daily, respectively.

What is claimed is:

1. A method of treating an inflammatory condition in an individual, comprising administering to the individual an effective amount of a composition comprising a compound having the structure of Formula I: 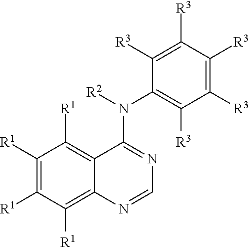
or a pharmaceutically acceptable salt thereof, wherein:
each R¹ is independently hydrogen, hydroxy, halogeno, amino, carboxy, carbamoyl, ureido, (1-4C)alkoxycarbonyl, N-(1-4C)alkylcarbamoyl, N,N-di-[(1-4C)alkyl]carbamoyl, hydroxyamino, (1-4C)alkyl, (1-4C)alkoxy, (1-3C)alkylenedioxy, (1-4C)alkylamino, di-[(1-4C)alkyl]amino, (1-4C)alkylthio, (1-4C)alkyl sulphinyl, (1-4C)alkylsulphonyl, bromomethyl, dibromomethyl, hydroxy-(1-4C)alkyl, (2-4C)alkanoyloxy-(1-4C)alkyl, (1-4C)alkoxy-(1-4C)alkyl, amino-(1-4C)alkyl, (1-4C)alkylamino-(1-4C)alkyl, di-[(1-4C)alkyl]amino-(1-4C)alkyl, piperidino-(1-4C)alkyl, morpholino-(1-4C)alkyl, piperazin-1-yl-(1-4C)alkyl, (1-4C)alkylpiperazin-1-yl-(1-4C)alkyl, hydroxy-(2-4C)alkoxy-(1-4C)alkyl, (1-4C)alkoxy-(2-4C)alkoxy(1-4C)alkyl, hydroxy-(2-4C)alkylamino-(1-4C)alkyl, (1-4C)alkoxy-(2-4C)alkylamino-(1-4C)alkyl, (1-4C)alkylthio-(1-4C)alkyl, hydroxy-(2-4C)alkylthio-(1-4C)alkyl, (1-4C)alkoxy-(2-4C)alkylthio-(1-4C)alkyl, cyano-(1-4C)alkyl, halogeno-(2-4C)alkoxy, hydroxy-(2-4C)alkoxy, (2-4C)alkanoyloxy-(2-4C)alkoxy, (1-4C)alkoxy-(2-4C)alkoxy, carboxy-(1-4C)alkoxy, (1-4C)alkoxycarbonyl-(1-4C)alkoxy, carbamoyl-(1-4C)alkoxy, N-(1-4C)alkylcarbamoyl-(1-4C)alkoxy, N,N-di-[(1-4C)alkyl]carbamoyl-(1-4C)alkoxy, amino-(2-4C)alkoxy, (1-4C)alkylamino-(2-4C)alkoxy, di-[(1-4C)alkyl]amino-(2-4C)alkoxy, piperidino-(2-4C)alkoxy, morpholino-(2-4C)alkoxy, piperazin-1-yl-(2-4C)alkoxy, (1-4C)alkylpiperazin-1-yl-(2-4C)alkoxy, halogeno-(2-4C)alkylamino, hydroxy-(2-4C)alkylamino, (2-4C)alkanoyloxy-(2-4C)alkylamino, (1-4C)alkoxy-(2-4C)alkylamino, amino-(2-4C)alkylamino, (1-4C)alkylamino-(2-4C)alkylamino, di-[(1-4C)alkyl]amino-(2-4C)alkylamino, (2-4C)alkanoylamino, (1-4C)alkyl sulphonylamino, benzamido, benzenesulphonamido, 2-oxopyrrolidin-1-yl, 2,5-dioxopyrrolidin-1-yl, halogeno-(2-4C)alkanoylamino, hydroxy-(2-4C)alkanoylamino, (1-4C)alkoxy-(2-4C)alkanoylamino, carboxy-(2-4C)alkanoylamino, (1-4C)alkoxycarbonyl-(2-4C)alkanoylamino, amino-(2-4C)alkanoylamino, (1-4C)alkylamino-(2-4C)alkanoylamino or di-[(1-4C)alkyl]amino-(2-4C)alkanoylamino, and wherein said benzamido or benzenesulphonamido substituent may optionally bear one or two halogeno, (1-4C)alkyl or (1-4C)alkoxy substituents,
or two R¹ groups are taken together with the carbons to which they are attached to form a 5-15 membered ring that includes 1-6 heteroatoms selected from oxygen, sulfur, and nitrogen;
R² is hydrogen or (1-4C)alkyl;
each R³ is independently hydrogen, hydroxy, halogeno, trifluoromethyl, amino, nitro, cyano, (1-4C)alkyl, (1-4C)alkoxy, H(2-4C)alkynl, or (2-4C)alkenyl;
wherein at least two R¹ are hydrogen;
wherein at least three R³ are hydrogen; and
wherein when Formula I is Formula III, the inflammatory condition is not psoriasis.

2. A method of treating type 2 diabetes in an individual, comprising administering to the individual an effective amount of a composition comprising a compound having the structure of Formula I: 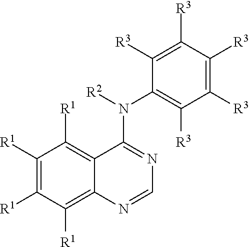
or a pharmaceutically acceptable salt thereof, wherein:
each R¹ is independently hydrogen, hydroxy, halogeno, amino, carboxy, carbamoyl, ureido, (1-4C)alkoxycarbonyl, N-(1-4C)alkylcarbamoyl, N,N-di-[(1-4C)alkyl]carbamoyl, hydroxyamino, (1-4C)alkyl, (1-4C)alkoxy, (1-3C)alkylenedioxy, (1-4C)alkylamino, di-[(1-4C)alkyl]amino, (1-4C)alkylthio, (1-4C)alkyl sulphinyl, (1-4C)alkylsulphonyl, bromomethyl, dibromomethyl, hydroxy-(1-4C)alkyl, (2-4C)alkanoyloxy-(1-4C)alkyl, (1-4C)alkoxy-(1-4C)alkyl, amino-(1-4C)alkyl, (1-4C)alkylamino-(1-4C)alkyl, di-[(1-4C)alkyl]amino-(1-4C)alkyl, piperidino-(1-4C)alkyl, morpholino-(1-4C)alkyl, piperazin-1-yl-(1-4C)alkyl, (1-4C)alkylpiperazin-1-yl-(1-4C)alkyl, hydroxy-(2-4C)alkoxy-(1-4C)alkyl, (1-4C)alkoxy-(2-4C)alkoxy(1-4C)alkyl, hydroxy-(2-4C)alkylamino-(1-4C)alkyl, (1-4C)alkoxy-(2-4C)alkylamino-(1-4C)alkyl, (1-4C)alkylthio-(1-4C)alkyl, hydroxy-(2-4C)alkylthio-(1-4C)alkyl, (1-4C)alkoxy-(2-4C)alkylthio-(1-4C)alkyl, cyano-(1-4C)alkyl, halogeno-(2-4C)alkoxy, hydroxy-(2-4C)alkoxy, (2-4C)alkanoyloxy-(2-4C)alkoxy, (1-4C)alkoxy-(2-4C)alkoxy, carboxy-(1-4C)alkoxy, (1-4C)alkoxycarbonyl-(1-4C)alkoxy, carbamoyl-(1-4C)alkoxy, N-(1-4C)alkylcarbamoyl-(1-4C)alkoxy, N,N-di-[(1-4C)alkyl]carbamoyl-(1-4C)alkoxy, amino-(2-4C)alkoxy, (1-4C)alkylamino-(2-4C)alkoxy, di-[(1-4C)alkyl]amino-(2-4C)alkoxy, piperidino-(2-4C)alkoxy, morpholino-(2-4C)alkoxy, piperazin-1-yl-(2-4C)alkoxy, (1-4C)alkylpiperazin-1-yl-(2-4C)alkoxy, halogeno-(2-4C)alkylamino, hydroxy-(2-4C)alkylamino, (2-4C)alkanoyloxy-(2-4C)alkylamino, (1-4C)alkoxy-(2-4C)alkylamino, amino-(2-4C)alkylamino, (1-4C)alkylamino-(2-4C)alkylamino, di-[(1-4C)alkyl]amino-(2-4C)alkylamino, (2-4C)alkanoylamino, (1-4C)alkyl sulphonylamino, benzamido, benzenesulphonamido, 2-oxopyrrolidin-1-yl, 2,5-dioxopyrrolidin-1-yl, halogeno-(2-4C)alkanoylamino, hydroxy-(2-4C)alkanoylamino, (1-4C)alkoxy-(2-4C)alkanoylamino, carboxy-(2-4C)alkanoylamino, (1-4C)alkoxycarbonyl-(2-4C)alkanoylamino, amino-(2-4C)alkanoylamino, (1-4C)alkylamino-(2-4C)alkanoylamino or di-[(1-4C)alkyl]amino-(2-4C)alkanoylamino, and wherein said benzamido or benzenesulphonamido substituent may optionally bear one or two halogeno, (1-4C)alkyl or (1-4C)alkoxy substituents,
or two R¹ groups are taken together with the carbons to which they are attached to form a 5-15 membered ring that includes 1-6 heteroatoms selected from oxygen, sulfur, and nitrogen;
R² is hydrogen or (1-4C)alkyl;
each R³ is independently hydrogen, hydroxy, halogeno, trifluoromethyl, amino, nitro, cyano, (1-4C)alkyl, (1-4C)alkoxy, H(2-4C)alkynl, or (2-4C)alkenyl;
wherein at least two R¹ are hydrogen; and
wherein at least three R³ are hydrogen.

3. The method of claim 2, further comprising identifying an individual in need of treatment for type 2 diabetes prior to the administration of the effective amount of a composition comprising a compound having the structure of Formula I.

4. A method of treating multiple sclerosis in an individual, comprising administering to the individual an effective amount of a composition comprising a compound having the structure of Formula I: 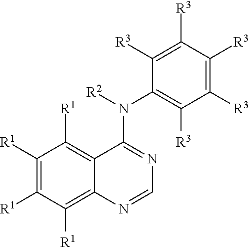
or a pharmaceutically acceptable salt thereof, wherein:
each R¹ is independently hydrogen, hydroxy, halogeno, amino, carboxy, carbamoyl, ureido, (1-4C)alkoxycarbonyl, N-(1-4C)alkylcarbamoyl, N,N-di-[(1-4C)alkyl]carbamoyl, hydroxyamino, (1-4C)alkyl, (1-4C)alkoxy, (1-3C)alkylenedioxy, (1-4C)alkylamino, di-[(1-4C)alkyl]amino, (1-4C)alkylthio, (1-4C)alkyl sulphinyl, (1-4C)alkylsulphonyl, bromomethyl, dibromomethyl, hydroxy-(1-4C)alkyl, (2-4C)alkanoyloxy-(1-4C)alkyl, (1-4C)alkoxy-(1-4C)alkyl, amino-(1-4C)alkyl, (1-4C)alkylamino-(1-4C)alkyl, di-[(1-4C)alkyl]amino-(1-4C)alkyl, piperidino-(1-4C)alkyl, morpholino-(1-4C)alkyl, piperazin-1-yl-(1-4C)alkyl, (1-4C)alkylpiperazin-1-yl-(1-4C)alkyl, hydroxy-(2-4C)alkoxy-(1-4C)alkyl, (1-4C)alkoxy-(2-4C)alkoxy(1-4C)alkyl, hydroxy-(2-4C)alkylamino-(1-4C)alkyl, (1-4C)alkoxy-(2-4C)alkylamino-(1-4C)alkyl, (1-4C)alkylthio-(1-4C)alkyl, hydroxy-(2-4C)alkylthio-(1-4C)alkyl, (1-4C)alkoxy-(2-4C)alkylthio-(1-4C)alkyl, cyano-(1-4C)alkyl, halogeno-(2-4C)alkoxy, hydroxy-(2-4C)alkoxy, (2-4C)alkanoyloxy-(2-4C)alkoxy, (1-4C)alkoxy-(2-4C)alkoxy, carboxy-(1-4C)alkoxy, (1-4C)alkoxycarbonyl-(1-4C)alkoxy, carbamoyl-(1-4C)alkoxy, N-(1-4C)alkylcarbamoyl-(1-4C)alkoxy, N,N-di-[(1-4C)alkyl]carbamoyl-(1-4C)alkoxy, amino-(2-4C)alkoxy, (1-4C)alkylamino-(2-4C)alkoxy, di-[(1-4C)alkyl]amino-(2-4C)alkoxy, piperidino-(2-4C)alkoxy, morpholino-(2-4C)alkoxy, piperazin-1-yl-(2-4C)alkoxy, (1-4C)alkylpiperazin-1-yl-(2-4C)alkoxy, halogeno-(2-4C)alkylamino, hydroxy-(2-4C)alkylamino, (2-4C)alkanoyloxy-(2-4C)alkylamino, (1-4C)alkoxy-(2-4C)alkylamino, amino-(2-4C)alkylamino, (1-4C)alkylamino-(2-4C)alkylamino, di-[(1-4C)alkyl]amino-(2-4C)alkylamino, (2-4C)alkanoylamino, (1-4C)alkyl sulphonylamino, benzamido, benzenesulphonamido, 2-oxopyrrolidin-1-yl, 2,5-dioxopyrrolidin-1-yl, halogeno-(2-4C)alkanoylamino, hydroxy-(2-4C)alkanoylamino, (1-4C)alkoxy-(2-4C)alkanoylamino, carboxy-(2-4C)alkanoylamino, (1-4C)alkoxycarbonyl-(2-4C)alkanoylamino, amino-(2-4C)alkanoylamino, (1-4C)alkylamino-(2-4C)alkanoylamino or di-[(1-4C)alkyl]amino-(2-4C)alkanoylamino, and wherein said benzamido or benzenesulphonamido substituent may optionally bear one or two halogeno, (1-4C)alkyl or (1-4C)alkoxy substituents,
or two R¹ groups are taken together with the carbons to which they are attached to form a 5-15 membered ring that includes 1-6 heteroatoms selected from oxygen, sulfur, and nitrogen;
R² is hydrogen or (1-4C)alkyl;
each R³ is independently hydrogen, hydroxy, halogeno, trifluoromethyl, amino, nitro, cyano, (1-4C)alkyl, (1-4C)alkoxy, H(2-4C)alkynl, or (2-4C)alkenyl;
wherein at least two R¹ are hydrogen; and
wherein at least three R³ are hydrogen.

5. The method of claim 2, wherein:
each R¹ is independently hydrogen, (1-4C)alkoxy, (1-4C)alkoxy-(2-4C)alkoxy, or morpholino-(2-4C)alkoxy,
or two R¹ groups are taken together with the carbons to which they are attached to form a 5-15 membered ring that includes 1-6 heteroatoms selected from oxygen, sulfur, and nitrogen;
R² is hydrogen; and
R³ is hydrogen, halogeno, or H(2-4C)alkynl.

6. The method of claim 2, wherein the composition is administered for a period of at least about 4 weeks.

7. The method of claim 2, wherein the composition is administered for a period of at least about 10 weeks.

8. The method of claim 2, wherein the composition is administered for a period of at least about 24 weeks.

9. The method of claim 2, wherein the composition is in oral dosage form.

10. The method of claim 9, wherein the composition is in a sustained-release formulation.

11. The method of claim 9, wherein the daily dosage of the compound is about 50 mg to about 200 mg per day.

12. The method of claim 11, wherein the daily dosage of the compound is about 150 mg per day.

13. The method of claim 2, wherein the compound has the structure of Formula II: 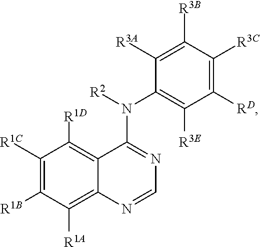
wherein R^{1A} and R^{1D} are hydrogen;
wherein R^{1B} and R^{1C} are methoxyethoxy, methoxy, morpholino-4-yl-propoxy, or wherein R^{1B} and R^{1C} are taken together with the carbons to which they are attached to form a 12 membered ring that includes 4 oxygen heteroatoms;
wherein R^{3A}, R^{3B}, and R^{3E} are hydrogen;
wherein R^{3C} is hydrogen or halogeno; and
wherein R^{3D} is halogeno or ethynyl.

14. The method of claim 13, wherein R^{1B} is methoxy.

15. The method of claim 13, wherein R^{1C} is morpholino-4-yl-propoxy.

16. The method of claim 13, wherein R^{1B} and R^{1C} are methoxyethoxy.

17. The method of claim 13, wherein R^{3C} is hydrogen.

18. The method of claim 13, wherein R^{3C} is halogeno.

19. The method of claim 18, wherein R^{3C} is fluorine.

20. The method of claim 13, wherein R^{3D} is halogeno.

21. The method of claim 200, wherein R^{3D} is chlorine.

22. The method of claim 13, wherein R^{3D} is ethynyl.

23. The method of claim 13, wherein R^{1B} and R^{1C} are taken together with the carbons to which they are attached to form a 12 membered ring that includes 4 oxygen heteroatoms.

24. The method of claim 2, wherein the compound has the structure of Formula III or a pharmaceutically acceptable salt thereof:

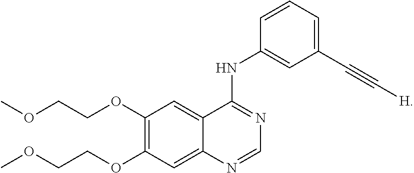

25. The method of claim 2, wherein the compound has the structure of Formula IV or a pharmaceutically acceptable salt thereof:

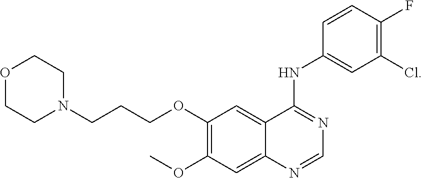 [SMILES: COc1cc2ncnc(Nc3ccc(F)c(Cl)c3)c2cc1OCCCN1CCOCC1]

26. The method of claim 2, wherein the compound has the structure of Formula V or a pharmaceutically acceptable salt thereof:

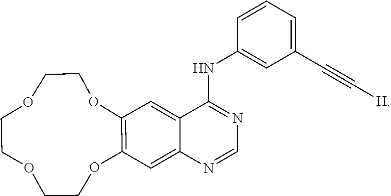

27. A method of evaluating a compound for the treatment of an inflammatory condition comprising contacting TNF-α with a compound and evaluating the results, wherein the compound has the structure of Formula I: 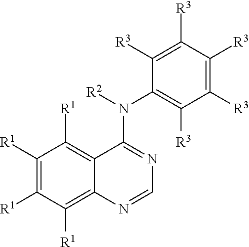
or a pharmaceutically acceptable salt thereof, wherein:
each R¹ is independently hydrogen, hydroxy, halogeno, amino, carboxy, carbamoyl, ureido, (1-4C)alkoxycarbonyl, N-(1-4C)alkylcarbamoyl, N,N-di-[(1-4C)alkyl]carbamoyl, hydroxyamino, (1-4C)alkyl, (1-4C)alkoxy, (1-3C)alkylenedioxy, (1-4C)alkylamino, di-[(1-4C)alkyl]amino, (1-4C)alkylthio, (1-4C)alkyl sulphinyl, (1-4C)alkylsulphonyl, bromomethyl, dibromomethyl, hydroxy-(1-4C)alkyl, (2-4C)alkanoyloxy-(1-4C)alkyl, (1-4C)alkoxy-(1-4C)alkyl, amino-(1-4C)alkyl, (1-4C)alkylamino-(1-4C)alkyl, di-[(1-4C)alkyl]amino-(1-4C)alkyl, piperidino-(1-4C)alkyl, morpholino-(1-4C)alkyl, piperazin-1-yl-(1-4C)alkyl, (1-4C)alkylpiperazin-1-yl-(1-4C)alkyl, hydroxy-(2-4C)alkoxy-(1-4C)alkyl, (1-4C)alkoxy-(2-4C)alkoxy(1-4C)alkyl, hydroxy-(2-4C)alkylamino-(1-4C)alkyl, (1-4C)alkoxy-(2-4C)alkylamino-(1-4C)alkyl, (1-4C)alkylthio-(1-4C)alkyl, hydroxy-(2-4C)alkylthio-(1-4C)alkyl, (1-4C)alkoxy-(2-4C)alkylthio-(1-4C)alkyl, cyano-(1-4C)alkyl, halogeno-(2-4C)alkoxy, hydroxy-(2-4C)alkoxy, (2-4C)alkanoyloxy-(2-4C)alkoxy, (1-4C)alkoxy-(2-4C)alkoxy, carboxy-(1-4C)alkoxy, (1-4C)alkoxycarbonyl-(1-4C)alkoxy, carbamoyl-(1-4C)alkoxy, N-(1-4C)alkylcarbamoyl-(1-4C)alkoxy, N,N-di-[(1-4C)alkyl]carbamoyl-(1-4C)alkoxy, amino-(2-4C)alkoxy, (1-4C)alkylamino-(2-4C)alkoxy, di-[(1-4C)alkyl]amino-(2-4C)alkoxy, piperidino-(2-4C)alkoxy, morpholino-(2-4C)alkoxy, piperazin-1-yl-(2-4C)alkoxy, (1-4C)alkylpiperazin-1-yl-(2-4C)alkoxy, halogeno-(2-4C)alkylamino, hydroxy-(2-4C)alkylamino, (2-4C)alkanoyloxy-(2-4C)alkylamino, (1-4C)alkoxy-(2-4C)alkylamino, amino-(2-4C)alkylamino, (1-4C)alkylamino-(2-4C)alkylamino, di-[(1-4C)alkyl]amino-(2-4C)alkylamino, (2-4C)alkanoylamino, (1-4C)alkyl sulphonylamino, benzamido, benzenesulphonamido, 2-oxopyrrolidin-1-yl, 2,5-dioxopyrrolidin-1-yl, halogeno-(2-4C)alkanoylamino, hydroxy-(2-4C)alkanoylamino, (1-4C)alkoxy-(2-4C)alkanoylamino, carboxy-(2-4C)alkanoylamino, (1-4C)alkoxycarbonyl-(2-4C)alkanoylamino, amino-(2-4C)alkanoylamino, (1-4C)alkylamino-(2-4C)alkanoylamino or di-[(1-4C)alkyl]amino-(2-4C)alkanoylamino, and wherein said benzamido or benzenesulphonamido substituent may optionally bear one or two halogeno, (1-4C)alkyl or (1-4C)alkoxy substituents,
or two R¹ groups are taken together with the carbons to which they are attached to form a 5-15 membered ring that includes 1-6 heteroatoms selected from oxygen, sulfur, and nitrogen;
R² is hydrogen or (1-4C)alkyl;
each R³ is independently hydrogen, hydroxy, halogeno, trifluoromethyl, amino, nitro, cyano, (1-4C)alkyl, (1-4C)alkoxy, H(2-4C)alkynl, or (2-4C)alkenyl;
wherein at least two R¹ are hydrogen; and
wherein at least three R³ are hydrogen.

28. The method of claim, 27, wherein said inflammatory condition is type 2 diabetes or multiple sclerosis.
